(19) United States
(12) Patent Application Publication
(10) Pub. No.: US 2026/0263229 A1
(43) Pub. Date: Sep. 10, 2026

(54) ORTHOPEDIC HUMERAL TRAY AND BEARING DESIGNS AND RELATED INSTRUMENTS

(71) Applicant: Zimmer, inc., Warsaw, IN (US)

(72) Inventors: Kevin Miller, Wakarusa, IN (US); Donald W. Dye, JR., Warsaw, IN (US); Christopher A Gray, Warsaw, IN (US); Maged Awadalla, Warsaw, IN (US); Cody Johnathon Holmes, Warsaw, IN (US)

(21) Appl. No.: 19/491,348

(22) PCT Filed: Jun. 17, 2024
(86) PCT No.: PCT/US2024/034363

§ 371 Date: Dec. 8, 2025

Related U.S. Application Data
(60) Provisional application No. 63/522,316, filed on Jun. 21, 2023.
(60) Provisional application No. 63/529,044, filed on Jul. 26, 2023.

(51) Int. Cl. A61F 2/40; A61F 2/30; A61F 2/46

(52) U.S. Cl. CPC A61F 2/4014; A61F 2/4637; A61F 2/4684; A61F 2002/30616

(57) ABSTRACT

A prosthesis assembly for a reverse shoulder arthroplasty that includes a bearing (1320) having a tab (1332) arranged along at least a first part of an outer edge thereof and a toe projection (1335) arranged along a second part of an outer edge thereof. The prosthesis assembly further includes a humeral implant (1322) having a wall with a barb (1348) and an insertion slot (1352) passing through the wall at a location distal of the barb, wherein the insertion slot is configured to receive a portion of an insertion tool therein, and wherein a portion of the barb engages with the tab coupling the bearing with the humeral implant.

### CLAIM OF PRIORITY

This application claims the benefit of U.S. Provisional Patent Application Ser. No. 63/522,316, filed on Jun. 21, 2023, and also claims the benefit of U.S. Provisional Patent Application Ser. No. 63/529,044, filed on Jul. 26, 2023, the benefit of priority of each of which is claimed hereby, and each of which is incorporated by reference herein in its entirety.

### FIELD OF THE DISCLOSURE

The present disclosure relates to an orthopedic shoulder prostheses that can be used during an arthroplasty procedure and to instruments that support the installation, sizing and removal of such prostheses.

### BACKGROUND

In the human body, tissue can require repair and replacement. Such tissue includes bone, muscles, tendons, ligaments and cartilage. For example, disease can necessitate replacement of bone(s) of the joint with one or more prosthetic components. Such replacement can require use of orthopedic guides and other instruments to facilitate proper orientation and/or size of the one or more prosthetic components.

The human shoulder joint may require repair or replacement. A conventional or reverse joint replacement may be used in a situation where the bone is diseased and/or a rotator cuff is damaged or lacking. This can provide pain relief and return the shoulder joint to normal kinematic function (e.g., the patient can again raise their arm above their head).

### SUMMARY

The present disclosure provides orthopedic prostheses including humeral implants and bearings and systems that include such prostheses and supporting instruments that can be used in the shoulder arthroplasty. Components such as the bearing are shown configured for a reverse shoulder arthroplasty. However, the concepts discussed can be applied to components such as the bearing used in an anatomic shoulder arthroplasty. Additionally, the concepts discussed herein can be extended to other prostheses and instruments and to other joints of the human body, and thus, can be applicable to orthopedic prostheses and instruments for the hip, knee, ankle, etc.

The present inventors have realized humeral implants and bearings that are more compact in size thereby reducing the chances of impingement with soft tissue and/or bone. More compact designs are also easier to implant in the patient, are more anatomically appropriate for smaller patient anatomy, and can result in a reduced trauma to the joint as compared with relatively larger designs. However, the present inventors have realized that more compact designs for the humeral implant and bearing can create challenges with regard to coupling these components together, decoupling the components from one another, and maintaining the components coupled together in a durable manner.

The present inventors have described herein a humeral bearing design that utilizes outward flexing tabs to couple with mating locking features on the humeral implant. These outward flexing tabs allow more robust coupling locking features to be used with the humeral implant, thereby, a size of the humeral implant can be reduced. Further benefits are recognized by the present inventors and include that the bearing can be configured to be rotated/clocked to a plurality of desired positions based upon the patient's anatomy with accuracy and reference to a neutral angle/position. The humeral implant can include a skirt, which can discourage soft tissue or bone debris from entering the spaced between the humeral implant and the bearing. Yet the skirt can accommodate the outward flexing tabs of the bearing. The present inventors also describe instruments that facilitate coupling and decoupling of the bearing with the humeral implant in a more streamlined, timely and easily replicated manner. Thus, surgical complexity and procedure time is reduced. The present inventors also describe a trial component for the bearing, which can be easily manipulated and installed by the surgeon when performing sizing/kinematics. Again, this can reduce time and surgical complexity related to these aspects of the orthopedic procedure.

The above discussion is intended to provide an overview of subject matter of the present patent application. It is not intended to provide an exclusive or exhaustive explanation of the invention. The description below is included to provide further information about the present patent application To better illustrate the orthopedic apparatuses and orthopedic systems disclosed herein, a non-limiting list of examples is provided here:

In Example 1 is a prosthesis assembly for a reverse shoulder arthroplasty optionally including a bearing and a humeral implant. The bearing can have a plurality of fingers arranged along at least a first part of an outer edge thereof. The humeral implant can have a wall with a plurality of receptacles configured to receive the plurality of fingers, wherein a number of the plurality of receptacles exceeds a number of the plurality of fingers allowing the bearing to be clocked relative to the humeral implant at a plurality of desired angles.

In Example 2, the prosthesis assembly of Example 1, wherein optionally the bearing includes plurality of tabs that form a second part of an outer edge of the bearing, wherein the plurality of fingers are arranged along a lateral side of the bearing and the plurality of tabs are arranged along a medial side of the bearing.

In Example 3, the prosthesis assembly of Example 2, wherein optionally the each of the plurality of tabs are separated by at least a first relief and the plurality of tabs are separated from the plurality of fingers by a second relief and a third relief, wherein the plurality of tabs each have a barb that extends inward toward a centerline axis of the bearing, wherein the humeral implant having a barb adjacent to and proximal of a groove, the barb extending outward away from a centerline axis of the humeral implant, wherein the plurality of tabs are configured to flex outward over the barb of the humeral implant to be at least partially received in the groove when the bearing is coupled to the humeral implant.

In Example 4, the prosthesis assembly of Example 3, wherein optionally one of the second relief and the third relief is configured to receive a portion of the wall when the bearing is clocked relative to the humeral implant at the plurality of desired angles.

In Example 5, the prosthesis assembly of Example 3, wherein optionally the humeral implant includes a skirt positioned distal of the groove, wherein the skirt projects outward of the groove away from a centerline axis of the humeral implant.

In Example 6, the prosthesis assembly of any one of Examples 1-5, further optionally comprising a plurality of indicia arranged on the bearing and the humeral implant to indicate the plurality of desired angles of the bearing relative to the humeral implant.

In Example 7, the prosthesis assembly of Example 6, wherein optionally the plurality of desired angles include at least a neutral angle relative to a scapular plane, an angle of between 15 and 45 degrees clockwise relative to the neutral angle, and an angle of between 15 and 45 degrees counterclockwise relative to the neutral angle.

Example 8 is a method of assembling a prosthesis for a reverse shoulder arthroplasty, the method optionally including: arranging a bearing at a desired angle/position relative to a humeral tray including by aligning indicia corresponding to a plurality of fingers of the bearing to be received in at least some of a plurality of receptacles of the humeral tray; engaging one or more medial edge portions of an articular surface and/or an outer edge of the bearing; and forcing down the bearing via engagement with the one or more medial edge portions onto the humeral tray such that a plurality of tabs of the bearing flex outward and pass over a corresponding mating feature of the humeral tray.

In Example 9, the method of Example 8, wherein optionally corresponding mating feature of the humeral tray includes a rail and a groove, and wherein the plurality of tabs engage the rail from a distal side with portion of the plurality of tabs also received in the groove.

In Example 10, the method of any one of Examples 8-9, wherein optionally the engaging can include inserting the plurality of fingers in at least some of the plurality of receptacles fully such that the plurality of fingers engage with a wall of the humeral bearing such that the bearing is coupled to the humeral bearing.

Example 11 is a prosthesis assembly for a reverse shoulder arthroplasty comprising: a bearing having a plurality of tabs that form a part of an outer edge thereof, each of the plurality of tabs separated by a relief, wherein the plurality of tabs each have a barb that extends inward toward a centerline axis of the bearing; and a humeral implant having a groove configured to receive the plurality of tabs, the humeral implant having a barb adjacent to and proximal of the groove, the barb extending outward away from a centerline axis of the humeral implant, wherein the plurality of tabs are configured to flex outward over the barb of the humeral implant to be at least partially received in the groove when the bearing is coupled to the humeral implant.

In Example 12, the subject matter of Example 11 optionally includes, wherein the humeral implant includes an alignment boss positioned at an outer perimeter of the humeral implant, wherein the alignment boss is configured to be received by the relief, wherein the relief comprises a plurality of reliefs at desired spaced intervals, wherein the plurality of reliefs allow the bearing to be clocked relative to the humeral implant at a plurality of desired angles.

In Example 13, the subject matter of Example 12 optionally includes, at least a neutral angle relative to a scapular plane, an angle of between 15 and 45 degrees clockwise relative to the neutral angle, and an angle of between 15 and 45 degrees counterclockwise relative to the neutral angle.

In Example 14, the subject matter of Examples 12-13 optionally includes, wherein the plurality of reliefs include a plurality of windows providing a larger opening to access portions of the groove and barb of the humeral implant, and wherein the humeral implant includes a plurality of detents within the groove at desired spaced intervals, wherein one or more of the plurality of detents are accessible via the plurality of windows.

In Example 15, the subject matter of Examples 11-14 optionally includes, wherein the humeral implant includes a skirt positioned distal of the groove, wherein the skirt projects outward of the groove away from the centerline axis of the humeral implant.

In Example 16, the subject matter of Example 15 optionally includes, wherein an outer surface of the plurality of tabs is positioned radially inward of an outer edge of the skirt when the bearing is coupled to the humeral implant.

In Example 17, the subject matter of Examples 11-16 optionally includes, wherein the relief includes a plurality of windows providing a larger opening to access portions of the groove and barb of the humeral implant, and wherein the humeral implant includes a plurality of detents within the groove at desired spaced intervals, wherein one or more of the plurality of detents are accessible via the plurality of windows.

In Example 18, the subject matter of Example 17 optionally includes, a bearing press having a clamp configured to engage one or more of the plurality of detents of the humeral implant and an actuator configured to engage the clamp and the bearing and force the bearing onto the humeral implant.

In Example 19, the subject matter of Examples 17-18 optionally includes, bearing tong press comprising a single component having a clamp configured to engage one or more of the plurality of detents of the humeral implant and an actuator configured to engage the clamp and the bearing and force the bearing onto the humeral implant.

In Example 20, the subject matter of Examples 17-19 optionally includes, a tool, wherein the plurality of windows are configured to provide access to the tool for insertion of the tool between the bearing and the humeral implant for removal of the bearing from the humeral implant.

In Example 21, the subject matter of Examples 11-20 optionally includes, wherein the bearing comprises a trial component has a one or more locking mechanisms separated from a main body by a plurality of reliefs including the relief, wherein bridges attach each of the one or more locking mechanisms to the main body, wherein the one or more locking mechanisms are flexed via the bridges outward to flex at least some of the plurality of tabs outward over the barb of the humeral implant to be received at least partially in the groove when the trial component is coupled to the humeral implant.

In Example 22, the subject matter of Example 21 optionally includes, wherein the trial component is configured to prevent the one or more locking mechanisms from flexing inward along a proximal articular portion when assembled on the humeral implant and engaged by a glenosphere, whereby the trial component is locked onto the humeral implant during a range of motion assessment.

In Example 23, the subject matter of Examples 11-22 optionally includes, a drill guide configured to mount on the bearing, wherein the drill guide includes at least one aperture for guiding drilling though the bearing to the humeral implant.

In Example 24, the subject matter of Examples 11-23 optionally includes, wherein the humeral implant comprises one of: a tray configured to couple with a stem via a mechanical locking mechanism or a single piece prosthesis.

Example 25 is an prosthesis assembly for a reverse shoulder arthroplasty optionally comprising: a trial component configured to simulate a bearing, wherein the trial component has a plurality of tabs that form an outer edge thereof, each of the plurality of tabs separated by a relief, wherein the trial component has a one or more locking mechanisms separated from a main body by a plurality of reliefs including the relief, wherein bridges attach each of the one or more locking mechanisms to the main body; and a humeral implant having a groove configured to receive the plurality of tabs, the humeral implant having a barb adjacent to and proximal of the groove, the barb extending outward away from a centerline axis of the humeral implant, wherein the one or more locking mechanisms are flexed via the bridges outward to flex at least some of the plurality of tabs outward over the barb of the humeral implant to be received at least partially in the groove when the trial component is coupled to the humeral implant.

Example 26 is an orthopedic system for a reverse shoulder arthroplasty optionally comprising: a bearing having a plurality of tabs that form an outer edge thereof, a humeral implant having a groove configured to receive the plurality of tabs, the humeral implant having a barb adjacent to and proximal of the groove, the barb extending outward away from a centerline axis of the humeral implant, wherein the plurality of tabs are configured to flex outward over the barb of the humeral implant to be at least partially received in the groove when the bearing is coupled to the humeral implant; and a trial component has a one or more locking mechanisms separated from a main body by a plurality of reliefs, wherein bridges attach each of the one or more locking mechanisms to the main body, wherein the one or more locking mechanisms are flexed via the bridges outward to flex at least some of the plurality of tabs outward over the barb of the humeral implant to be received at least partially in the groove when the bearing is coupled to the humeral implant.

In Example 27, the orthopedic system of Example 26, wherein the trial component optionally includes a plurality of windows defined by at least some of the plurality of tabs, wherein providing an opening to access portions of the groove and barb of the humeral implant, and wherein the humeral implant includes a plurality of detents within the groove at desired spaced intervals, wherein one or more of the plurality of detents are accessible via the plurality of windows.

In Example 28, the orthopedic system of any one of Examples 26-27, wherein optionally the trial component is configured to prevent the one or more locking mechanisms from flexing inward at a proximal articular portion when assembled on the humeral implant and engaged by a glenosphere, whereby the trial component is locked onto the humeral implant during a range of motion assessment.

In Example 29, the orthopedic system of any one of Examples 26-28, wherein the trial component optionally includes a skirt positioned distal of the groove, wherein the skirt projects outward of the groove away from the centerline axis of the humeral implant.

In Example 30, the orthopedic system of Example 29, wherein optionally an outer surface of the plurality of tabs is positioned radially inward of an outer edge of the skirt when the bearing is coupled to the humeral implant.

In Example 31, the orthopedic system of any one of Examples 26-30, wherein the trial component comprises a plurality of bearings of different configurations.

In Example 32, a trial component for a reverse shoulder arthroplasty optionally comprising a trial component configured to simulate a bearing. The trial component optionally has a one or more locking mechanisms separated from a main body by a plurality of reliefs. The bridges attach each of the one or more locking mechanisms to the main body. The one or more locking mechanisms can be configured to be flexed via the bridges for coupling one or more tabs to a humeral implant. The trial component can be configured to prevent the one or more locking mechanisms from flexing inward at a proximal articular portion when assembled on the humeral implant and engaged by a glenosphere.

In Example 33, the trial component of Example 32, wherein the plurality of reliefs optionally include edge reliefs and interior reliefs, wherein the interior reliefs communicate with an articular surface of the trial component.

In Example 34, the trial component of any one of Examples 32-33, wherein optionally the one or more locking mechanisms are configured to be manually flexed outward to flex at least some of the one or more tabs outward over portions of the humeral implant.

Example 35 is a prosthesis assembly for a reverse shoulder arthroplasty optionally comprising: a bearing having a tab arranged along at least a first part of an outer edge thereof and a toe projection arranged along a second part of an outer edge thereof; and a humeral implant having a wall with a barb and an insertion slot passing through the wall at a location distal of the barb, wherein the insertion slot is configured to receive a portion of an insertion tool therein, and wherein a portion of the barb engages with the tab coupling the bearing with the humeral implant.

In Example 36, the subject matter of Example 35 optionally includes, wherein the bearing includes at least a first relief and a second relief, wherein the first relief is configured to receive the toe projection adjacent the tab, and wherein the second relief is positioned outward of and proximal of the toe projection and is configured to receive the portion of the barb therein.

In Example 37, the subject matter of Example 36 optionally includes, wherein the bearing that includes tab forms a medial side of the bearing, and wherein the toe projection is arranged along a lateral side of the bearing.

In Example 38, the subject matter of Example 36 optionally includes, wherein the humeral implant includes a groove along a medial side thereof, wherein the insertion slot communicates with the groove.

In Example 39, the subject matter of Example 38 optionally includes, wherein the humeral implant includes a skirt positioned distal of the groove, wherein the skirt projects outward of the groove away from a centerline axis of the humeral implant, wherein the outer edge of the bearing along at least the first part is substantially aligned with or positioned inward of the skirt.

In Example 40, the subject matter of Example 39 optionally includes, wherein an opposing wall of the humeral implant on the lateral side includes a relief forming an undercut configured to receive the toe projection therein.

Example 41 is a prosthesis assembly for a reverse shoulder arthroplasty optionally comprising: a bearing having a tab arranged along at least a first part of an outer edge thereof; and a humeral implant having a wall with a barb and a skirt positioned distal of the barb, wherein the skirt projects outward of the tab away from a centerline axis of the humeral implant, wherein the tab is substantially aligned with or positioned inward of the skirt, and wherein a portion of the barb engages with the tab coupling the bearing with the humeral implant.

In Example 42, the subject matter of Example 41 optionally includes, an insertion slot passing through the wall at a location distal of the barb, wherein the insertion slot is configured to receive a portion of an insertion tool therein, and wherein a portion of the barb engages with the tab coupling the bearing with the humeral implant.

In Example 43, the subject matter of Example 42 optionally includes, a toe projection arranged along a second part of an outer edge thereof.

In Example 44, the subject matter of Example 43 optionally includes, wherein the bearing includes at least a first relief and a second relief, wherein the first relief is configured to receive the toe projection adjacent the tab, and wherein the second relief is positioned outward of and proximal of the toe projection and is configured to receive the portion of the barb therein.

In Example 45, the subject matter of Examples 41-44 optionally includes, wherein the humeral implant includes a groove along a medial side thereof, wherein the skirt is positioned distal of the groove, wherein the skirt projects outward of the groove away from a centerline axis of the humeral implant.

Example 46 is a prosthesis assembly for a reverse shoulder arthroplasty optionally comprising: a trial component configured to simulate a bearing, wherein the trial component has two coupling mechanisms including two arms each including at least one finger along an outer edge thereof, each of two arms separated by a relief, and a humeral implant configured to receive and couple with the trial component.

In Example 47, the subject matter of Example 46 optionally includes, wherein the humeral implant has a wall with a barb and an insertion slot passing through the wall at a location distal of the barb.

In Example 48, the subject matter of Example 47 optionally includes, wherein the humeral implant includes a groove along a medial side thereof adjacent the wall and barb, wherein the insertion slot communicates with the groove.

In Example 49, the subject matter of Example 48 optionally includes, wherein the humeral implant includes a skirt positioned distal of the groove, wherein the skirt projects outward of the groove away from a centerline axis of the humeral implant, wherein an outer edge of the trial component is substantially aligned with or positioned inward of the skirt.

In Example 50, the subject matter of Example 49 optionally includes, wherein an opposing wall of the humeral implant on a lateral side includes a relief forming an undercut configured to receive the two coupling mechanisms therein.

Example 51 is at least one machine-readable medium including instructions that, when executed by processing circuitry, cause the processing circuitry to perform operations to implement of any of Examples 1-50.

Example 52 is an apparatus comprising means to implement of any of Examples 1-50.

Example 53 is a method to implement of any of Examples 1-50.

In some aspects, the Examples described can include any one or combination of the apparatus and system examples above including any one or combination of the individual features disclosed herein.

### BRIEF DESCRIPTION OF THE FIGURES

The above-mentioned and other features and advantages of this disclosure, and the manner of attaining them, will become more apparent and the disclosure itself will be better understood by reference to the following description of examples taken in conjunction with the accompanying drawings, wherein:

FIG. 1 is an anatomic view of a shoulder joint of a patient.

FIG. 2 is a schematic diagram of an prosthesis assembly implanted in a humerus of the patient and forming at least a portion of the shoulder joint.

FIG. 3 is a perspective view of a bearing according to an example of the present application.

FIGS. 3A-3C are plan views of the bearing of FIG. 3.

FIG. 3D is a cross-sectional view of a portion of the bearing of FIG. 3 showing one of a plurality of tabs and other features according to an example of the present application.

FIG. 4 is a perspective view of an alternative bearing according to an example of the present application.

FIGS. 5A and 5B are a plan view and a perspective view of a humeral tray according to an example of the present application.

FIG. 6 is a humeral stem according to an example of the present application.

FIG. 7 is another example of the humeral stem according to the present application.

FIG. 8 is a perspective view of an inserter for assembling the humeral tray with the humeral stem according to an example of the present application.

FIG. 8A is an enlarged view of a distal end of the inserter engaging the humeral tray of FIG. 8.

FIGS. 9A-9C are views of the bearing of FIGS. 3-3D coupled to the humeral tray of FIGS. 5A and 5B according to an example of the present application.

FIG. 10 is a perspective view of the bearing of FIGS. 3-3D in the process of being coupled with the humeral tray with a plurality of tabs of the bearing being flexed outward over a barb or other feature of the humeral tray according to an example of the present application.

FIG. 11 is a perspective view of the bearing and humeral tray of FIG. 10 now coupled together according to an example of the present application.

FIG. 11A is an enlarged view of one of the plurality of tabs of the bearing of FIG. 11 engaging with features of the humeral tray including the barb and being partially received in a groove according to an example of the present application.

FIG. 12 shows a method and a bearing press and tray clamp that can be used to assemble the bearing to the humeral tray according to an example of the present application.

FIG. 13 are perspective views of a tong press that can be used to assemble the bearing to the humeral tray according to an example of the present application.

FIG. 14 are perspective views of an instrument such as an osteotome that can be utilized to disassemble the bearing from the humeral tray according to an example of the present application.

FIGS. 15A and 15B are perspective views of a drill guide that can be used for disassembling the bearing from the humeral tray according to an example of the present application.

FIGS. 16A and 16B are perspective views of a trial component that simulates the bearing according to an example of the present application.

FIGS. 17A and 17B show the trial component assembled with the humeral tray and clocked to different positions relative thereto according to an example of the present application.

FIGS. 18A-18B are perspective views of a humeral tray according to yet another example of the present application.

FIG. 18C is a plan view of the humeral tray of FIGS. 18A and 18B.

FIGS. 19A-19C are plan and perspective views of a bearing according to yet another example of the present application.

FIG. 20 is an enlarged view of part of a lateral side of an assembly of the humeral tray of FIGS. 18A-18C and the bearing of FIGS. 19A-19C according to an example of the present application.

FIG. 21 is an enlarged view of part of a medial side of an assembly of the humeral tray of FIGS. 18A-18C and the bearing of FIGS. 19A-19C according to an example of the present application.

FIGS. 22A and 22B are perspective views of the assembly of the of the humeral tray of FIGS. 18A-18C and the bearing of FIGS. 19A-19C according to an example of the present application.

FIG. 23 is a method of forming the assembly of FIGS. 20-22B.

FIGS. 24A-24E show various views of a second trial component that simulates the bearing of FIGS. 19A-19C according to an example of the present application.

FIGS. 25A and 25B are perspective views of a bearing according to an example of the present application.

FIGS. 26A and 26B are perspective views of another example of the bearing according to an example of the present application.

FIG. 27 is a perspective view of a humeral tray according to an example of the present application.

FIGS. 28A and 28B are perspective views of an orthopedic assembly of the bearing of FIGS. 26A and 26B coupled to the humeral tray of FIG. 27 according to an example of the present application.

FIG. 28C is a cross-sectional view of the orthopedic assembly of FIGS. 28A and 28B.

FIG. 29 is a perspective view of an inserter for assembling the humeral tray of FIG. 27 with the bearing of FIGS. 26A and 26B according to an example of the present application.

FIGS. 30A and 30B are perspective view and plan view of a trial component that simulates the bearing according to an example of the present application.

FIG. 31A shows a method of assembling the trial component of FIGS. 30A and 30B with the humeral tray of FIG. 27 according to an example of the present application.

FIG. 31B shows a method of disassembling the trial component of FIGS. 30A and 30B from the humeral tray of FIG. 27 according to an example of the present application.

FIG. 32 shows a tool configured for removal of the bearing of FIGS. 26A and 26B from the humeral tray of FIG. 27 according to an example of the present application.

FIG. 32A is an enlarged view of portions of the tool of FIG. 32 and the bearing of FIGS. 26A and 26B from the humeral tray of FIG. 27 according to an example of the present application.

Corresponding reference characters indicate corresponding parts throughout the several views. The exemplifications set out herein illustrate examples of the disclosure, and such exemplifications are not to be construed as limiting the scope of the disclosure any manner.

### DETAILED DESCRIPTION

In describing the examples of the disclosure illustrated and to be described with respect to the drawings, specific terminology will be used for the sake of clarity. However, the disclosure is not intended to be limited to any specific terms or illustrations used herein, and it is to be understood that each specific term includes all technical equivalents.

The present disclosure is directed to orthopedic apparatuses and orthopedic systems that can be used in joint replacement procedures such as a reverse shoulder arthroplasty. Although the present apparatuses and systems are being described in reference to a reverse shoulder arthroplasty, the apparatuses and systems can be used for other procedures and other joints as discussed previously.

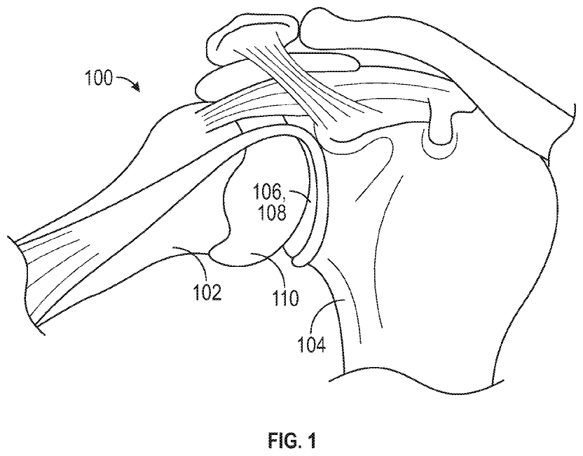

FIG. 1 illustrates a shoulder joint 100 with several ligaments stripped away. As shown, the shoulder joint 100 includes a humerus 102 and a scapula 104 that has a glenoid 106 with a socket 108 for interacting with a humeral head 110 of humerus 102. Humeral head 110 can articulate within a socket 108 to allow for normal motion of the shoulder joint 100. Disease can degenerate the bone or soft tissue of the humeral head 110 and/or scapula 104. These can cause pain and/or can negatively impact shoulder joint function. Typically, a surgical intervention may be required to replace the shoulder joint and restore shoulder joint function.

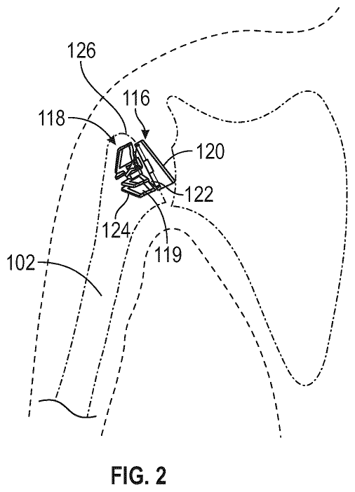

FIG. 2 illustrates a schematic diagram of a prosthesis assembly 116 installed at the shoulder joint 100. For simplicity, a corresponding prosthesis assembly installed in the scapula is not illustrated. The prosthesis assembly 116 can include a humeral implant 118 and a bearing 120. The humeral implant 118 can include a humeral tray 122 and a stem and/or stemless anchor 124. It is understood that in some examples the humeral implant 118 may not include the humeral tray and humeral stem as separate components but that these can be integrated together into a single component.

The humeral implant 118 can be fitted into a recess 119 formed at a proximal end portion 126 of the humerus 102. The embodiment of FIG. 2 show is a fin design for the stem and/or stemless anchor 124 that has a reduced length in a longitudinal direction. However, a more traditional stem design is also contemplated for the stem and/or stemless anchor 124. As shown in FIG. 2, the humeral tray 122 can interface with and can be coupled to the bearing 120 and the stem and/or stemless anchor 124. The bearing 120 can couple with the humeral tray 122 using locking mechanisms as further described herein. The concepts of the present application are not limited by the design examples for the bearing 120, the humeral tray 122 and the stem and/or stemless anchor 124 provided herein. Similarly, the example bone of the humerus should not be interpreted as limiting but merely exemplary.

Reverse total shoulder arthroplasty is one of several types of shoulder replacement surgeries. In reverse total shoulder arthroplasty, a portion of a patient's humerus and a portion of the patient's glenoid is replaced and/or augmented with implantable components. With a reverse shoulder arthroplasty, prosthesis components include a glenoid implant (not specifically shown in FIG. 2) that acts as the “ball” (semi-spherical or otherwise shaped to replicate the head of the humerus) and the bearing 120 shaped as a cup to receive the “ball”. The bearing 120 acts as the glenoid, in reverse shoulder arthroplasty, with the “ball” on the glenoid side of the shoulder joint. In other words, the relationship between the prosthesis components in the surgically created shoulder joint is opposite that of the anatomically correct shoulder joint.

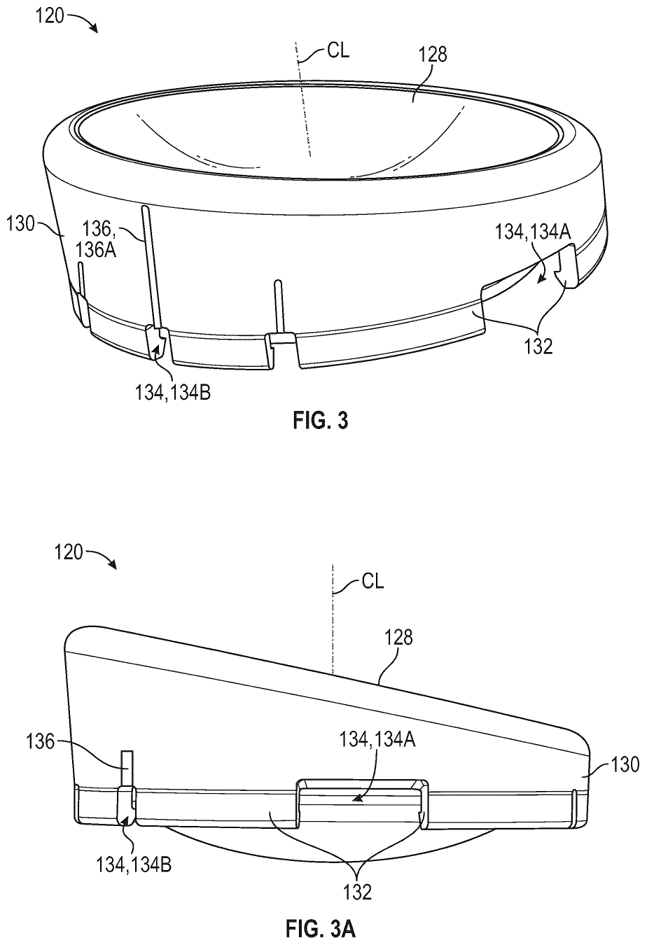

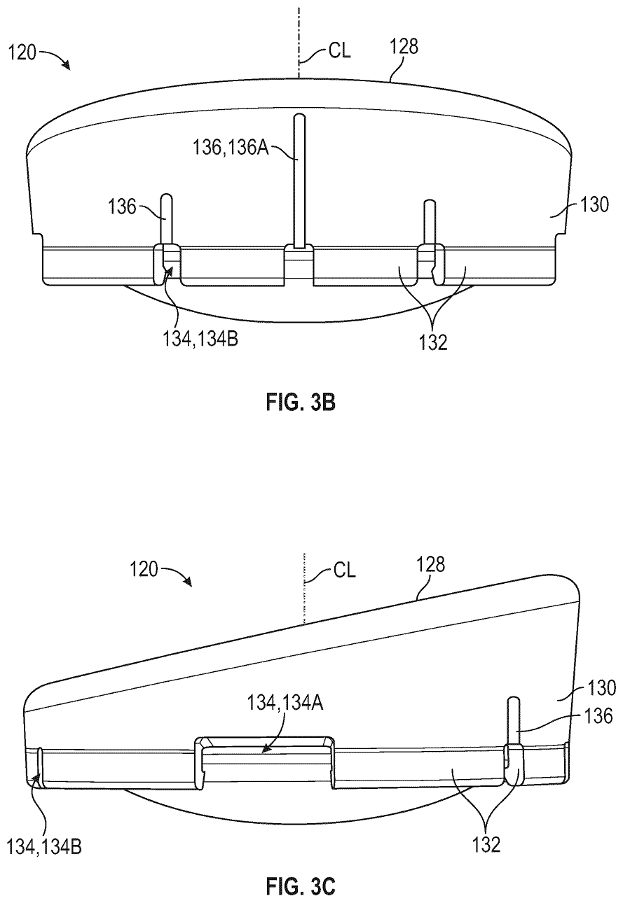

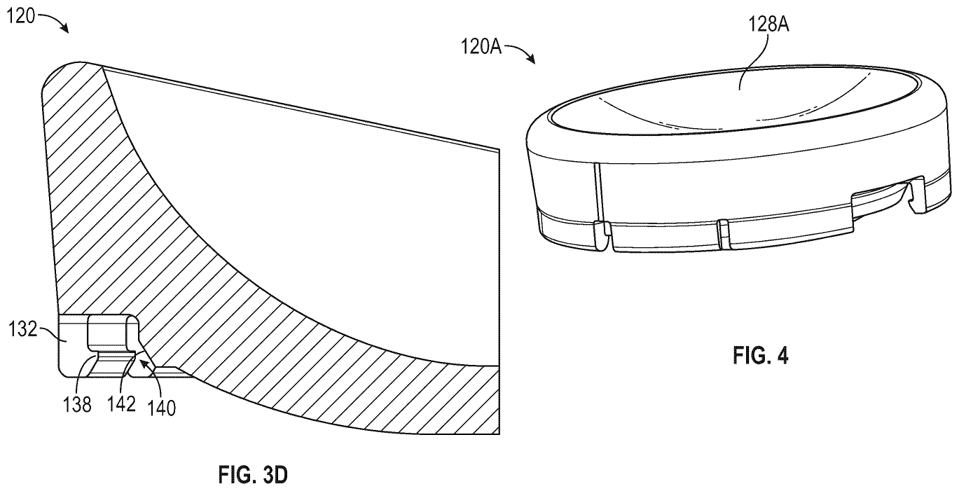

FIGS. 3-3C show an example of the bearing 120. The bearing 120 can include an articular surface 128, an outer edge 130 or side, a plurality of tabs 132, a plurality of reliefs 134 and indicia 136.

The bearing 120 can be formed of High Density Poly Ethylene (HDPE) or other suitable implantable material(s). The bearing 120 can be available in a range of standard sizes including different thicknesses and diameters. The bearing 120 can have a centerline axis CL. The articular surface 128 can form a proximal most side of the bearing 120 and can be cup or bowl shape to receive the ball component installed on the glenoid. The articular surface 128 can be angled or tilted according to some examples. Thus, the bearing 120 can have a varying thickness or generally sloped thickness from a medial side to a lateral side, for example. The articular surface 128 can extend to adjacent the outer edge 130. Outer edge 130 can form the side of the bearing 120. The outer edge 130 can extend distally from at or adjacent the articular surface 128 to the plurality of tabs 132. Outer sides of the plurality of tabs 132 can form part of the outer edge 130.

The plurality of tabs 132 can have lips, barb or another feature configured to engage the humeral implant (not shown) as further described and shown herein. The plurality of tabs 132 can be circumferentially arranged along a distal part of the bearing 120 forming a distal edge thereof. The plurality of tabs 132 can be spaced from one another by respective ones of the plurality of reliefs 134.

The plurality of reliefs 134 can comprise gaps between adjacent of the plurality of tabs 132. The plurality of reliefs 134 can include a plurality of windows 134A and alignment features 134B. The plurality of windows 134A can be relatively larger in size than the alignment features 134B. Like the plurality of reliefs 134, the plurality of windows 134A can be at least partially defined by some of the plurality of tabs 132. The alignment features 134B can have a desired spacing from one another as discussed subsequently. The indicia 136 can be positioned on the outer edge 130 adjacent and proximal of the alignment features 134B. The indicia 136 can be differently shaped such as including a relatively larger indicia 136A that indicates a neutral position/angle relative to a scapular plane.

FIG. 3D shows a cross-section of a portion of the bearing 120 particularly showing one of the plurality of tabs 132. As shown in FIG. 3D, each of the plurality of tabs 132 can include a barb 138 that extends inward toward the centerline axis CL (FIGS. 3-3C) of the bearing 120. The plurality of tabs 132 including the barb 138 can be spaced from an interior of the bearing 120 by a recess 140 or groove. A part of the interior of the bearing 120 can include a chamfered surface 142 designed to seat against and engage with the humeral tray as further described herein. The chamfered surface 142 can improve the assembly action by preventing binding when the bearing 120 and humeral tray aren't assembled in a directly axial direction.

FIG. 4 shows a second example of a bearing 120A of similar construction to the bearing 120 of FIGS. 3-3D but not including the angled or tilted design. Thus, the articular surface 128A is not angled or tilted and the bearing 120A can have a similar or same thickness at both a medial side as at a lateral side, for example.

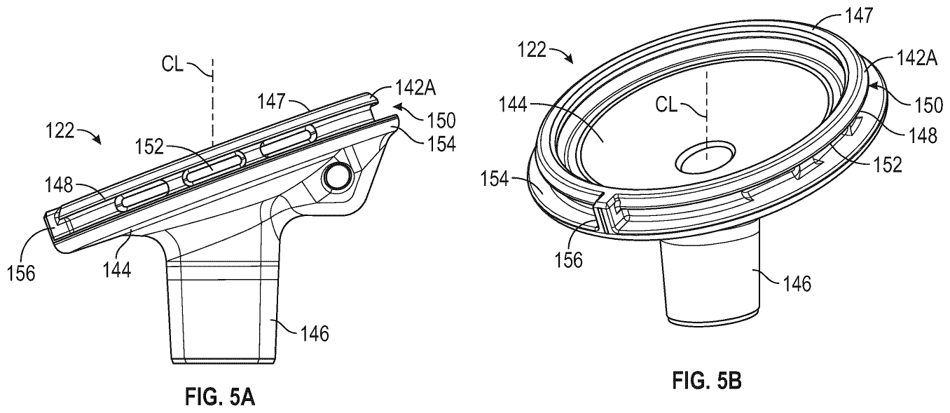

FIGS. 5A and 5B show the humeral tray 122. The humeral tray 122 can include a centerline axis CL, a main body 144, a stem 146, a rail 147, a barb 148, a groove 150, a plurality of detents 152, a skirt 154 and an alignment boss 156.

The main body 144 can be generally dish shaped or otherwise curved and configured to receive distal parts of the bearing (not shown). The stem 146 can extend distal of the main body 144 and can be configured with a taper or other mechanical feature to couple with the humeral stem (not shown). The rail 147 or other feature can extend from the main body 144 and can form an outer proximal edge of the humeral tray 122. The rail 147 can include a chamfered surface 142A configured to be engaged by the chamfered surface 142 of the bearing 120 (see FIG. 3D).

The barb 148 can project outward from the rail 147. The barb 148 can project generally outward from the centerline axis CL of the humeral tray 122. The barb 148 can extend around less than an entirety of a circumference of the humeral tray 122. The barb 148 can comprise an overhanging feature of the rail 147, for example. The barb 148 can form a portion of the groove 150.

The groove 150 can extend around a majority of the circumference of the humeral tray 122 distal of the barb 148. The plurality of detents 152 can be spaced from one another and can be positioned in the groove 150 at desired spaced intervals. The plurality of detents 152 can be recesses in the groove 150. The skirt 154 can be positioned distal of the groove 150 and can form a portion thereof. The skirt 154 can project radially outward of the groove 150, and indeed, radially outward of the barb 148 and rail 147. The alignment boss 156 can be positioned at an outer perimeter of the humeral tray 122. Although a single alignment boss (alignment boss 156) is illustrated, further examples contemplate the use of two or more alignment bosses at different spaced circumferential locations. The alignment boss 156 can extend from the skirt 154 and can project from the rail 147. The alignment boss 156 can be configured to be received in one of the plurality of reliefs 134 (e.g., the alignment features 134B of FIGS. 3-3C of the bearing 120) as further described and illustrated. The alignment boss 156 can include indicia, for example. The alignment boss 156 can be positioned on a medial-most side of the humeral tray 122, for example.

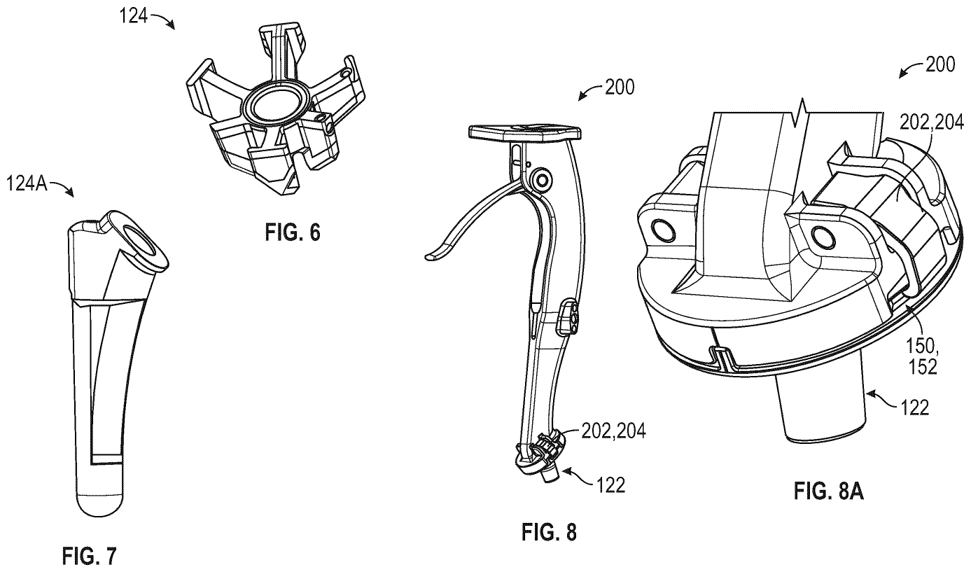

FIG. 6 shows the stem and/or stemless anchor 124, which can comprise a finned design according to one example. The stem and/or stemless anchor 124 can be configured to couple with the humeral tray 122 (FIGS. 5A and 5B) as further described and illustrated herein. As discussed, the stem and/or stemless anchor 124 can be configured to be implanted in the prepared recess of the proximal humerus. The design of the stem and/or stemless anchor 124 is sometimes described as a stemless or stem-free configuration. The stem and/or stemless anchor 124 can be configured in the manner of the Sidus® Stem-Free Shoulder prosthesis, Comprehensive® Nano Stemless Shoulder or another commercially available and manufactured by Zimmer Biomet Inc., of Warsaw Indiana. The humeral implant designs shown in FIGS. 6 and 7 are purely exemplary and are provided merely to facilitate practitioner understanding.

FIG. 7 shows a stem and/or stemless anchor 124A that can be utilized with the humeral tray 122 (FIGS. 5A and 5B) according to another example having an elongated length designed to insert within a prepared recess of the humerus. Other humeral stem designs not specifically shown are also contemplated.

FIG. 8 shows an inserter 200 according to one example. The inserter 200 can be configured to engage and couple with the humeral tray 122. FIG. 8A shows a locking mechanism 202 such as moveable jaws 204 can be configured to engage with the groove 150 (FIG. 8A) or some of the plurality of detents 152 (FIG. 8A) to couple the humeral tray 122 to the inserter 200. Using the inserter 200 the surgeon can insert the humeral tray 122 into the humeral stem (e.g., stem and/or stemless anchor 124, stem and/or stemless anchor 124A, etc.) to couple the humeral tray 122 to the humeral stem. The inserter 200 can be used to couple the humeral tray 122 to the humeral stem while the humeral stem is already implanted within the shoulder joint, for example. However, such process can also be performed at a back bench or other location.

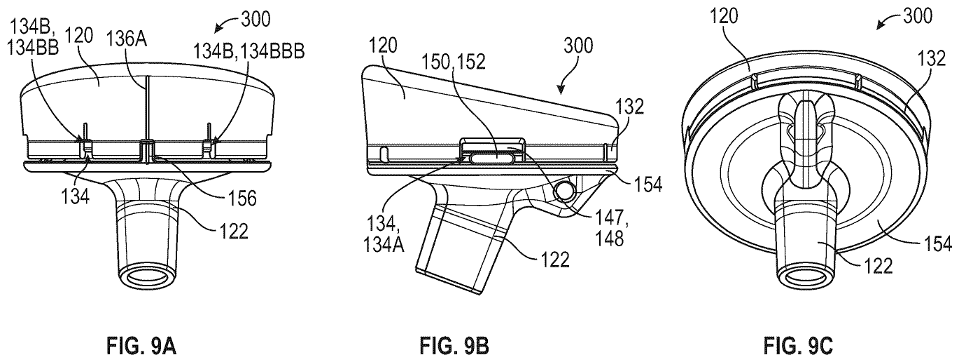

FIGS. 9A-9C show an orthopedic assembly 300 of the humeral tray 122 and the bearing 120. The bearing 120 is coupled to the humeral tray 122. FIG. 9A shows the alignment boss 156 received in one of the alignment features 134B (one of the plurality of reliefs 134). As shown in FIG. 9A, the bearing 120 is positioned at a neutral angle or position relative to humeral tray 122 with the indicia 136A aligned with the scapular plane. The alignment features 134BB and 134BBB (FIG. 9A) can be positioned clockwise or counterclockwise (e.g., at an angle) at a desired circumferential position relative to the alignment boss 156 when in the neutral position as shown in FIG. 9A. This angle can be between 15 and 45 degrees in the clockwise direction and between 15 and 45 degrees in the counterclockwise direction, for example. The angle in the clockwise direction can be the same as or can differ from the angle in the counterclockwise direction.

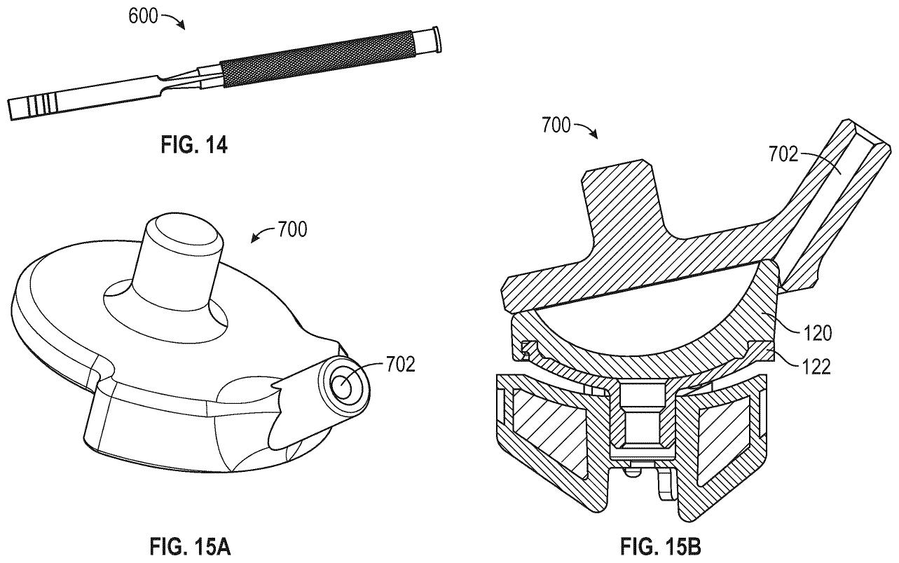

FIG. 9B shows one of the plurality of reliefs 134, in particular, one of the plurality of windows 134A positioned adjacent to and allowing access to one of the plurality of detents 152, parts of the rail 147, parts of the barb 148 and parts of the groove 150. A tool (illustrated in FIG. 14) can be inserted through the one of the plurality of windows 134A, which are configured to provide access to the tool for insertion of the tool between the bearing 120 and the humeral tray 122 for removal of the bearing 120 from the humeral tray 122.

As shown in FIGS. 9B and 9C, an outer surface of the plurality of tabs 132 is positioned radially inward of or flush with an outer edge of the skirt 154 when the bearing 120 is coupled to the humeral tray 122. The skirt 154 can extend to protect the plurality of tabs 132, the barb 148 and the groove 150 (together comprising a locking mechanism as shown in FIG. 9B) from contact with tissue.

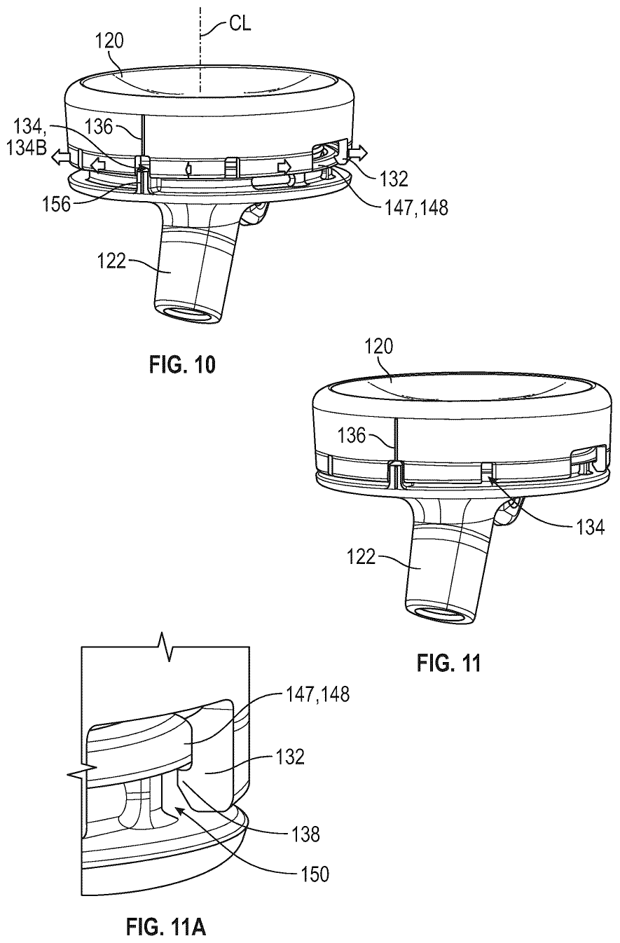

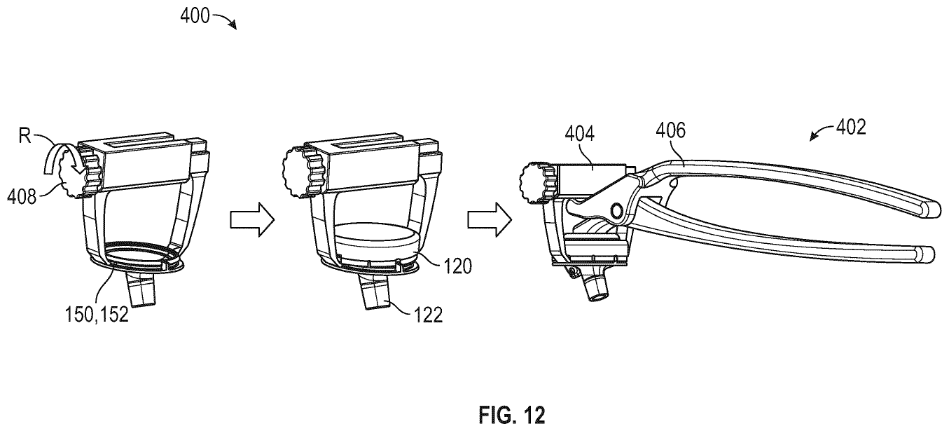

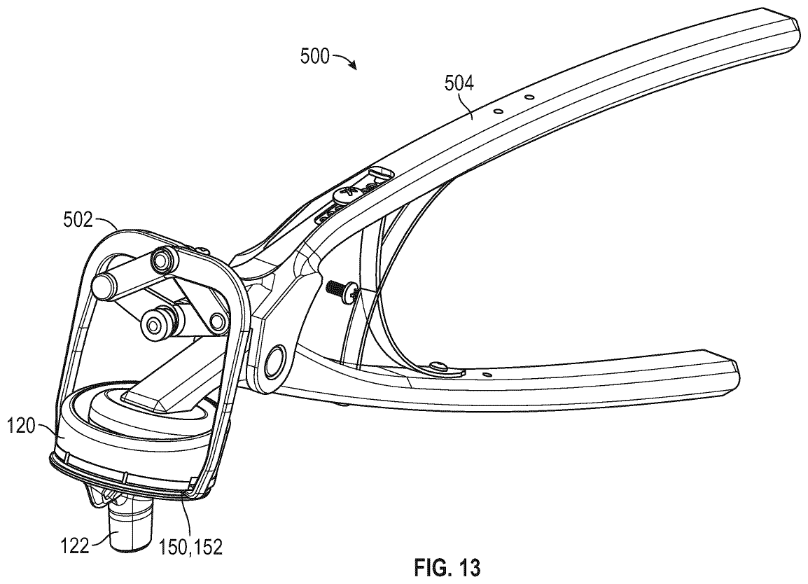

FIG. 10 illustrates the bearing 120 in the process of being coupled with the humeral tray 122. The alignment boss 156 can be aligned with one of the alignment features 134B. As shown in FIG. 10, the plurality of tabs 132 can be configured to flex outward (indicated with arrows) away from the centerline axis CL of the bearing 120 by engagement between the plurality of tabs 132 and portions of the rail 147 and/or barb 148. Thus, the plurality of tabs 132 are configured to flex outward over the barb 148 of the humeral tray 122 as shown in FIG. 10. The installation process of FIG. 10 can be accomplished by hand. However, assembly instruments such as those of FIGS. 12 and 13 are intended to provide a method for in-situ assembled when the patient may have poor bone quality and/or a fracture. The instruments provide a method for pressing the bearing into the tray without any reaction forces applied to the patient's bone.

FIGS. 11 and 11A show the bearing 120 coupled with the humeral tray 122. In such relative coupled position, the plurality of tabs 132 including the barb 138 are at least partially received in the groove 150 when the bearing 120 is coupled to the humeral tray 122 as shown in FIG. 11A. The edge snap configuration of the bearing 120 with the humeral tray 122 can provide an audible sound when the plurality of tabs 132 pass over the barb 148 and engage with the barb 148 on a distal side and are partially received in the groove 150. Additionally, the configuration of the bearing 120 with the plurality of reliefs 134 and indicia 136 can facilitate that alignment/clocking can be verified before and after coupling as shown in FIGS. 10 and 11. Visual confirmation that the bearing is seated is also provided by the now near-zero gap between the bearing and tray skirt.

FIG. 12 shows a method 400 of coupling the bearing 120 to the humeral tray 122 using a two component instrument assembly comprising a bearing press 402. The bearing press 402 includes a clamp 404 and an actuator 406. The clamp 404 can be configured to engage the groove 150 and/or the one or more of the plurality of detents 152 of the humeral tray 122. This process can be done by rotating a knob 408 as indicated by arrow R in the image to the viewer's left. The actuator 406 can be configured to engage the clamp 404 (when the clamp is engaged with the humeral tray 122) and the bearing 120 and force the bearing 120 onto the humeral tray 122 in the manner shown in FIGS. 10-11A and also shown in FIG. 12.

FIG. 13 shows a single piece instrument assembly comprising a bearing tong press 500 having a clamp 502 configured to engage the groove 150 and/or the one or more of the plurality of detents 152 of the humeral tray 122. The bearing tong press 500 can include an actuator 504 configured to engage the clamp 502 and the bearing 120 and force the bearing 120 onto the humeral tray 122 in the manner shown in FIGS. 10-11A and also shown in FIG. 13.

FIG. 14 shows a tool such as an osteotome 600 that can be used for removal of the bearing 120 from the humeral tray 122 (see FIG. 9B). Referring now to FIG. 9B, the osteotome 600 (FIG. 14) can be configured to insert through one or more of the plurality of windows 134A. Put another way, the plurality of windows 134A are configured to provide access to the osteotome 600 for insertion of the osteotome 600 between the bearing 120 and the humeral tray 122 for removal of the bearing 120 from the humeral tray 122.

FIGS. 15A and 15B show another tool, a drill guide 700, which can be used for removal of the bearing 120 from the humeral tray 122. The drill guide 700 can be configured to mount on the bearing 120 as shown in FIG. 15B. The drill guide 700 includes at least one aperture 702 for guiding drilling though the bearing 120 to the humeral tray 122. Once a hole through the bearing 120 has been formed, a fastener or other component (not shown) can be inserted into the drilled hole and forced against the humeral tray 122. The fastener can be rotated to push the bearing 120 off the humeral tray 122 to decouple the bearing 120 from the humeral tray 122.

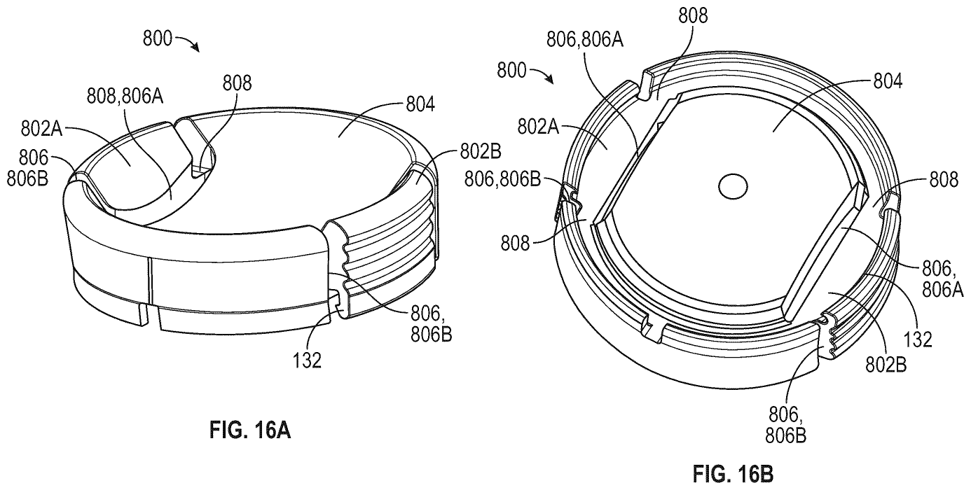

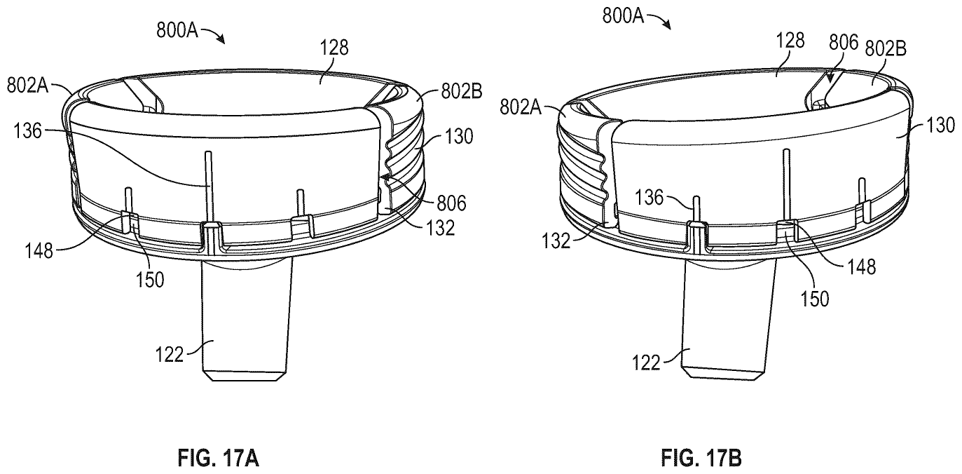

FIGS. 16A and 16B show a trial component 800. The trial component 800 can be configured to simulate the configuration (shape, size, position, etc.) of the bearing previously discussed. Thus, the trial component 800 can be available in a plurality of standard sizes and shapes, for example. The trial component 800 can be a temporary component that can be coupled to either the humeral tray 122 (see FIGS. 17A and 17B) previously discussed or a humeral tray trial. The trial component 800 can be used for sizing the bearing 120 (previously shown) and/or to perform joint kinematics such as range of motion. The trial component 800 has a plurality of locking mechanisms 802A and 802B separated from a main body 804. The plurality of locking mechanisms 802A and 802B can be separated from the main body 804 by a plurality of reliefs 806, including interior reliefs 806A and side reliefs 806B. Bridges 808 act as linkages to attach each of the plurality of locking mechanisms 802A and 802B to the main body 804 and extend between the interior reliefs 806A and the side reliefs 806B. The plurality of locking mechanisms 802A and 802B can be flexed via the bridges 808 outward to flex at least some of the plurality of tabs 132 outward.

FIGS. 17A and 17B show a different examples of a trial component 800A with a different shape from that of the trial component 800 of FIGS. 16A and 16B. Additionally, FIGS. 17A and 17B show the plurality of tabs 132 have been flexed outward by the plurality of locking mechanisms 802A and 802B and over the barb 148 of the humeral tray 122. This allows the plurality of tabs 132 to be received at least partially in the groove 150 when the trial component 800A is coupled to the humeral tray 122 as shown in FIGS. 17A and 17B. The trial component 800A (and indeed the trial component 800 of FIGS. 16A and 16B) can also include the previously described features including the articular surface 128, the outer edge 130 or side, the plurality of tabs 132, the plurality of reliefs 806 and indicia 136 in the manner discussed in regard to the bearing 120 (see FIGS. 3A-3D). During range of motion assessment, the trial component 800A (and indeed the trial component 800) has the articulating surface 128 assembled to a glenosphere, the plurality of locking mechanisms 802A and 802B (FIGS. 16A and 16B) are prevented from flexing inward at a proximal articular portion due to the configuration of the plurality of tabs 128, the bridges 808 (FIGS. 16A and 16B), the plurality of locking mechanisms 802A and 802B (FIGS. 16A and 16B) and the engagement and configuration of the glenosphere pressing against a proximal portion the plurality of locking mechanisms 802A and 802B along the articular surface. This configuration of the trial component 800 (and trial component 800) effectively locks the trial component 800A (and trial component 800) onto the tray during the range of motion assessment, where unexpected decoupling should be avoided.

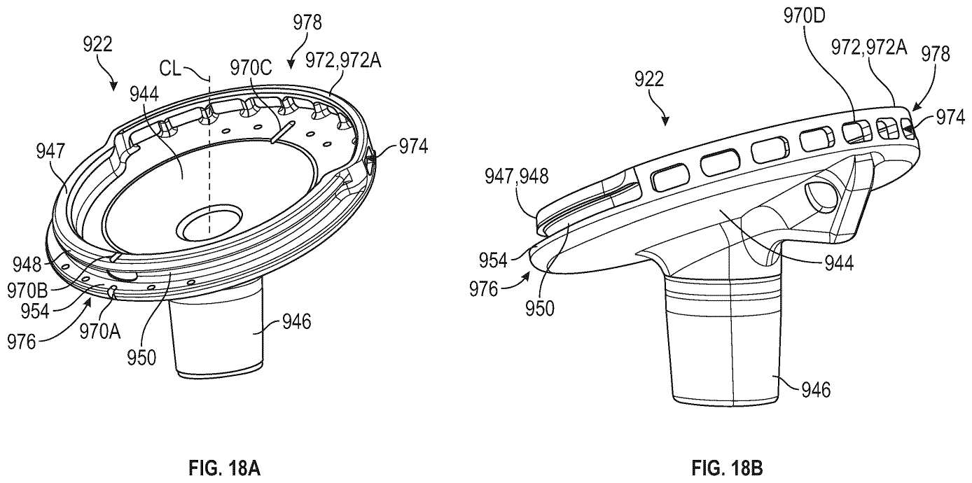

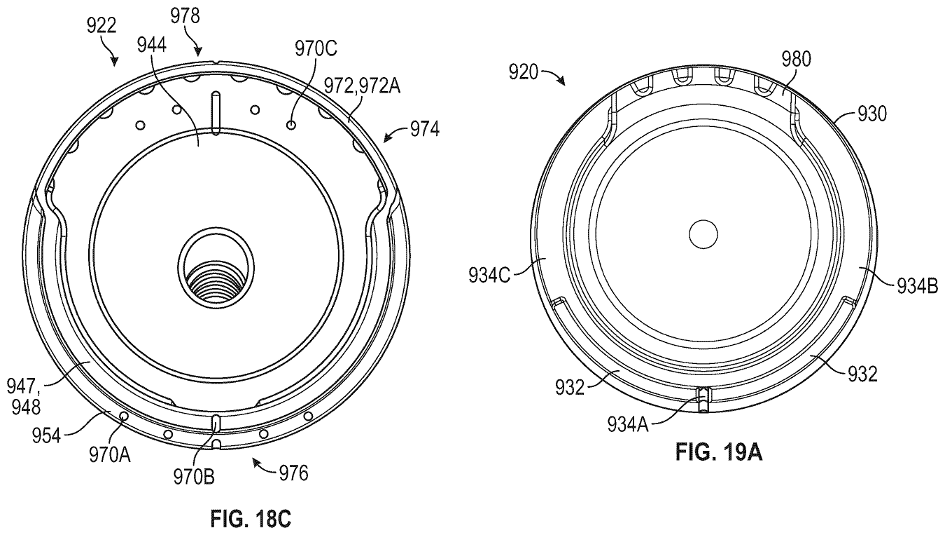

FIGS. 18A-18C show a humeral tray 922 according to another example. The humeral tray 922 can include features that differ from and features that are similar to that of the humeral tray 122 (FIGS. 5A and 5B). The humeral tray 922 can include a centerline axis CL (FIG. 18A only), a main body 944, a stem 946, a rail 947, a barb 948, a groove 950, a skirt 954, indicia 970A, 970B, 970C and 970D, a wall 972 and a plurality of receptacles 974.

The main body 944 can be generally dish shaped or otherwise curved and configured to receive distal parts of the bearing (not shown). The stem 946 can extend distal of the main body 944 and can be configured with a taper or other mechanical feature to couple with the humeral stem (not shown). The rail 947 can include the chamfered surface 142A configured to be engaged by a corresponding chamfered surface of the bearing as previously discussed. The rail 947 or other feature can extend proximally from a medial side 976 of the main body 144 and can form an outer medial proximal edge of the humeral tray 122.

The rail 947 can be connected to the wall 972 at a first end and a second end. The rail 947 can have a semi-circular or other extent about the outer proximal edge. The wall 972 can be arranged to project outward of the rail 947 with respect to the centerline axis CL. This is because the wall 972 does not have the skirt 954 adjacent therewith. The wall 972 can extend proximally from a lateral side 978 of the main body 944 and can form an outer lateral proximal edge and an a lateral outer circumference of the humeral tray 122. The wall 972 can have a semi-circular or other extend about the outer proximal edge. Wall 972 can have a proximal rail 972A. The plurality of receptacles 974 can be formed by the wall 972 adjacent but distal of the proximal rail 972A. The plurality of receptacles 974 can be spaced at increments from one another in a predefined manner (e.g., one receptacle every 10 degrees, one every 15 degrees, etc.) about a circumference of the wall 972.

The barb 948 can project outward from the rail 947 and can have a construction similar to the barb 148 (FIGS. 5A and 5B) discussed previously. Thus, the barb 948 can project generally outward from the centerline axis CL of the humeral tray 922. The barb 948 can extend around less than an entirety of a circumference of the humeral tray 922 as rail 947 extends around less than an entirety. The barb 948 can comprise an overhanging feature such as a projection of the rail 947, for example. The barb 948 can form a portion of the groove 950.

The groove 950 can extend around half or less (or in some cases more than half) of the circumference of the humeral tray 922 distal of the barb 948. The skirt 954 can be positioned distal of the groove 950 on the medial side 976 and can form a portion of the groove 950. The skirt 954 can project radially outward of the groove 950, and indeed, radially outward of the barb 948 and rail 947.

Referring to FIGS. 18A and 18C, the indicia 970A, 970B and 970C can be located at different positions on the humeral tray 922. The indicia 970A can be located on the skirt 954, for example and can be notches, dimples, lines, etc. The indicia 970A can indicate a neutral position and clocked (angled) positions relative to the neutral position. The indicia 970B can be located on a proximal or outside surface of the rail 947 and can indicate a medial most position and/or the neutral position. The indicia 970C can be located on a proximal surface of the main body 944 and can indicate the neutral position and clocked (angled) positions relative to the neutral position. Turning to FIG. 18B, the indicia 970D can be on the wall 972 such as along an outer circumference thereof.

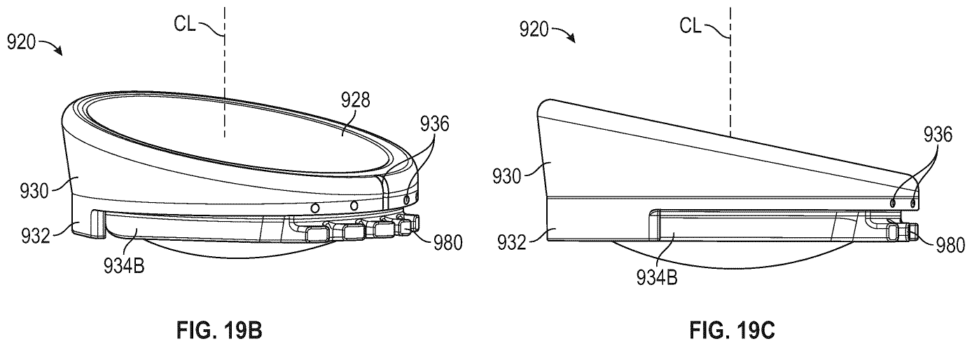

FIGS. 19A-19C show a bearing 920 according to another example. The bearing 120 can include an articular surface 928 (FIG. 19B only), an outer edge 930 or side, a plurality of tabs 932, a plurality of reliefs 934A, 934B and 934C, indicia 936 (FIGS. 19B and 19C) and a plurality of fingers 980.

FIG. 19A shows a distal side of the bearing 920 that is configured for coupling with the humeral tray 922 (FIGS. 18A-18C). The bearing 920 in FIG. 19A includes the plurality of tabs 932, the plurality of reliefs 934A, 934B and 934C and the plurality of fingers 980.

The bearing 920 can be formed of High Density Poly Ethylene (HDPE) or other suitable implantable material(s). The bearing 920 can be available in a range of standard sizes including different thicknesses and diameters. The bearing 920 can have a centerline axis CL (FIG. 19A only). The articular surface 928 can form a proximal most side of the bearing 920 and can be cup or bowl shape to receive the ball component installed on the glenoid. The articular surface 928 can be angled or tilted according to some examples. Thus, the bearing 920 can have a varying thickness or generally sloped thickness from a medial side to a lateral side, for example. The articular surface 928 can extend to adjacent the outer edge 930. Outer edge 930 can form the side of the bearing 920. The outer edge 930 can extend distally from at or adjacent the articular surface 928 (FIG. 19B) to the plurality of tabs 932. Outer sides of the plurality of tabs 932 can form part of the outer edge 930.

The plurality of tabs 932 can have lips, barb or another feature configured to engage the humeral implant (not shown) as further described and shown herein. The plurality of tabs 932 can be circumferentially arranged along a medial distal part of the bearing 920 forming a distal edge thereof. The plurality of tabs 932 can be spaced from one another by one or more of the plurality of reliefs 934A. The plurality of tabs 932 are spaced from the plurality of fingers 980 by respective ones of the plurality of reliefs 934A.

The plurality of reliefs 934A can comprise gaps such as between adjacent of the plurality of tabs 932 and also the plurality of reliefs 934B and 934C can be gaps between the plurality of tabs 932 and the plurality of fingers 980. The plurality of fingers 980 can have a desired spacing from one another matching/corresponding to the spacing of at least some of the plurality of receptacles 974 (FIGS. 18A-18C). The indicia 936 can be positioned on the outer edge 930 such as adjacent and proximal of the plurality of fingers 980 on the lateral side and/or adjacent one or more of the plurality of reliefs 934A on the medial side. The indicia 936 can be differently shaped such as including a relatively larger indicia 936A that indicates a neutral position/angle relative to a scapular plane.

The plurality of fingers 980 can be projections extending outward from the centerline axis CL from the plurality of reliefs 934B and 934C. The plurality of fingers 980 can be discrete projections and can be arranged along a lateral side of the bearing 920 at or adjacent the outer edge 930.

The plurality of tabs 932 can be constructed in the manner of the plurality of tabs 132 (FIGS. 3A-3D) discussed previously. However, the plurality of tabs 932 can have a lesser extent (e.g., can be positioned around half or less than half of the outer edge 930 and can be arranged along a medial side of the bearing 920. The plurality of tabs 932 can include the barb as discussed previously, with the barb spaced from an interior of the bearing 920 by a recess 940 or groove (see FIG. 19A).

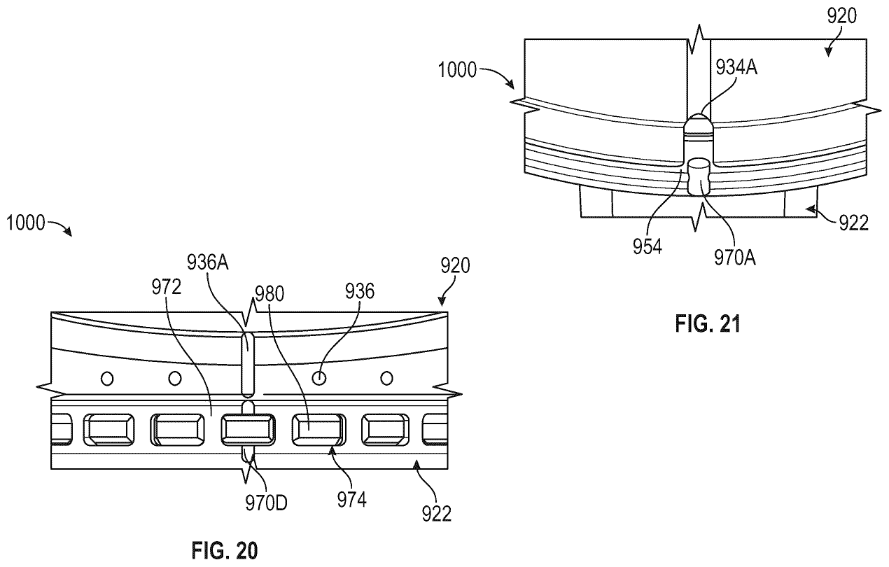

FIG. 20 shows a lateral side of an assembly 1000 with the bearing 920 engaging the humeral tray 922, the plurality of fingers 980 can be shaped such as with tapering or other features to engage with the wall 972 of the humeral tray 922 after a portion of the plurality of fingers 980 is inserted in the plurality of receptacles 974. The plurality of fingers 980 can be configured to create a press-fit or other type of engagement with the wall 972 when fully inserted into the plurality of receptacles 974. The number of the plurality of receptacles 974 can exceed the number of the plurality of fingers 980. This allows for the rotation/clocking of the bearing 920 to a desired angle relative to the humeral tray 922 as previously discussed. As an example, the plurality of fingers 980 can be selectively positioned clockwise or counterclockwise (e.g., at an angle) at a desired circumferential position relative to the neutral position causing different of the plurality of fingers 980 to be inserted into different of the plurality of receptacles 974. This angle can be between 15 and 45 degrees in the clockwise direction and between 15 and 45 degrees in the counterclockwise direction, for example. The angle in the clockwise direction can be the same as or can differ from the angle in the counterclockwise direction. FIG. 20 shows the indicia 936 including the indicia 936A (for neutral position) positioned along the outer edge 930 of the bearing 920 adjacent the wall 972, the plurality of fingers 980 and at least some of the plurality of receptacles 974. The indicia 936 can be aligned with the indicia 970D along the wall 972 and can correspond to the position of one or more of the plurality of fingers 980.

FIG. 21 shows a medial side of the assembly 1000 of the bearing 920 and the humeral tray 922 with one of the plurality of reliefs 934A aligned with the indicia 970A located on the proximal surface and/or outer surface of the skirt 954. This alignment indicates the bearing 920 is arranged in the neutral position relative to the humeral tray 922.

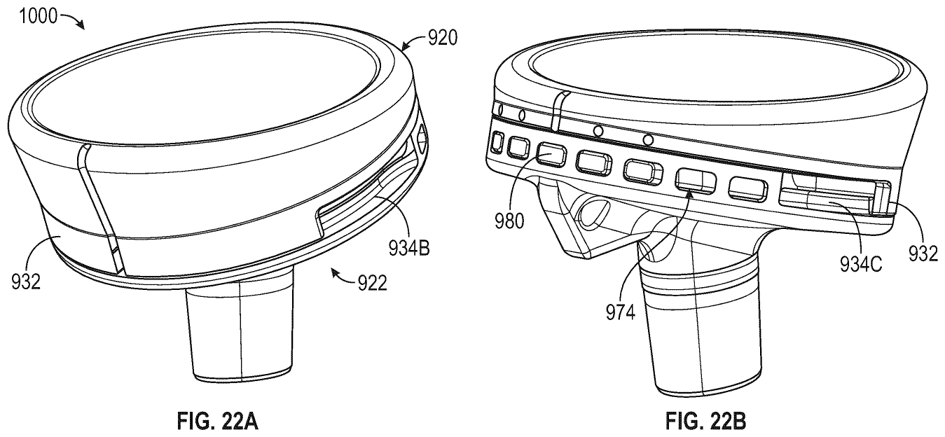

FIGS. 22A and 22B show the assembly 1000 of the bearing 920 and the humeral tray 922 coupled together. As shown in FIG. 22B, the plurality of fingers 980 are received in less than all of the plurality of receptacles 974. Additionally, the relief 934B and/or 934C is large enough in spacing between the last of the plurality of fingers 980 and the plurality of tabs 932 to accommodate the rotation of the bearing 920 relative to the humeral tray 922 to allow for the clocking to the desired angle as discussed previously.

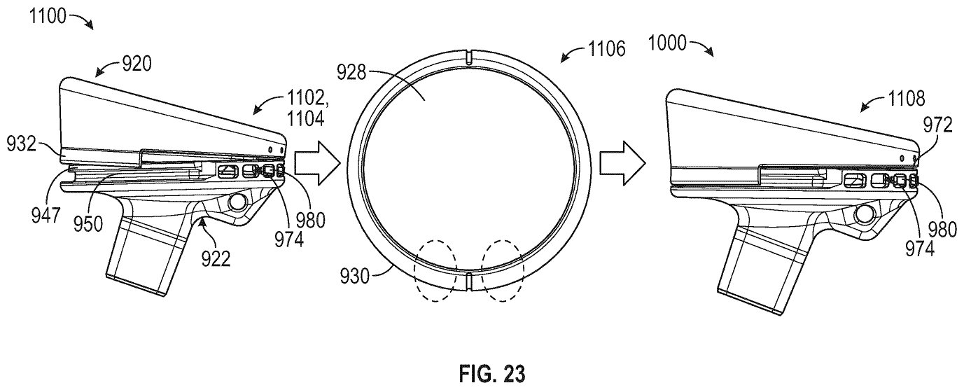

FIG. 23 shows a method 1100 of coupling the bearing 920 to the humeral tray 922 to form the assembly 1000 previously shown. The method 1100 can include arranging 1102 the bearing 920 at a desired angle/position relative to the humeral tray 922. This can include aligning indicia or other features as discussed herein including aligning 1104 the plurality of fingers 980 to be received in at least some of the plurality of receptacles 974. The method 1100 can include engaging 1106 one or more medial edge portions (indicated with ovals) of the articular surface 928 and/or outer edge 930 of the bearing 920 with hand or instrument. The bearing 920 on the medial side can be forced down (e.g., by via engagement with the one or more medial edge portions) such that the plurality of tabs 932 flex outward and pass over the rail 947 and engage the rail 947 from a distal side with portion of the tabs 932 received in the groove 950 as previously described and illustrated therein. The method 1100 of engaging 1108 can include inserting the plurality of fingers 980 in at least some of the plurality of receptacles 974 fully such that the plurality of fingers 980 engage with the wall 972 such that the bearing 920 is coupled to the humeral tray 922 and cannot be readily decoupled without the use of the instruments discussed previously.

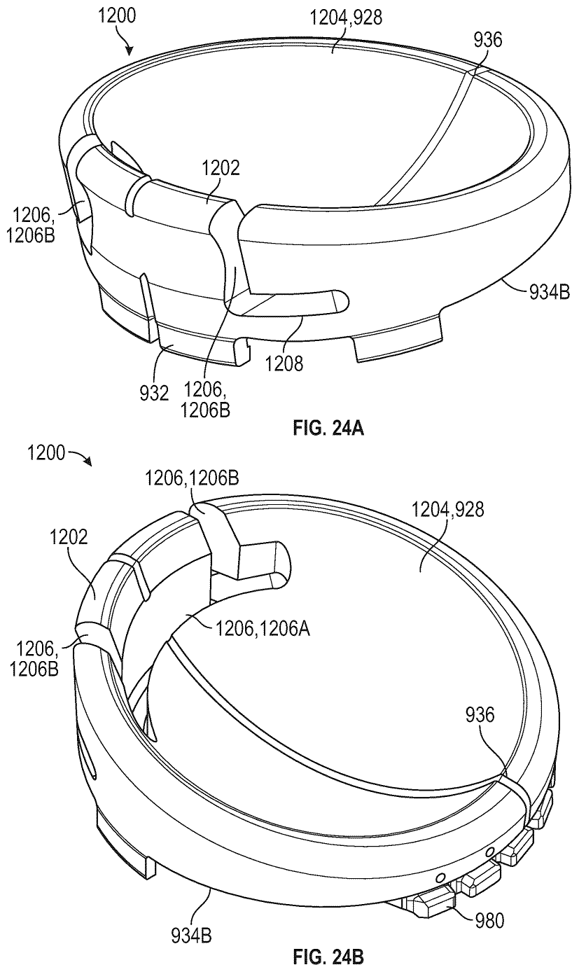

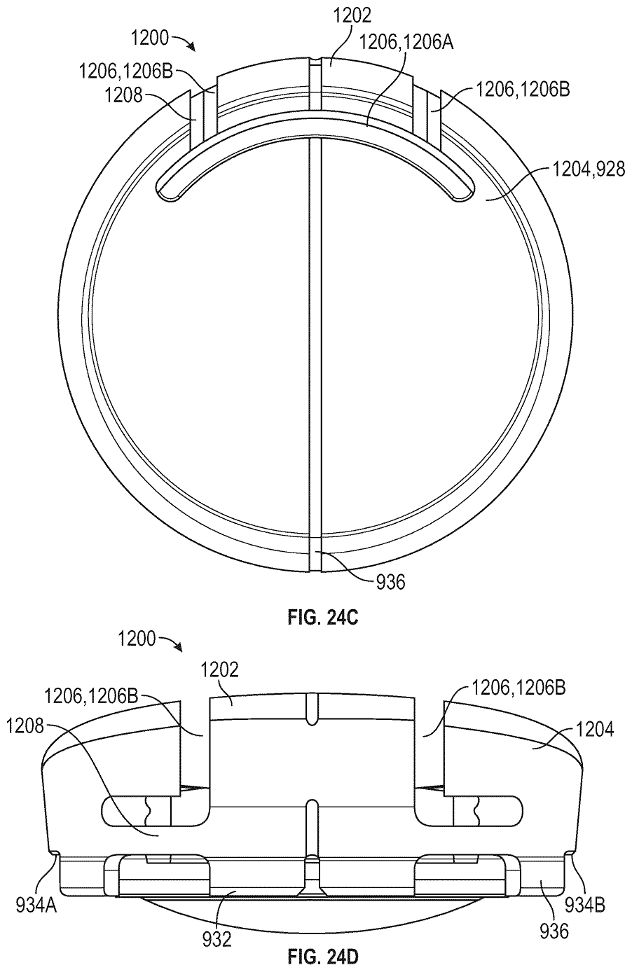

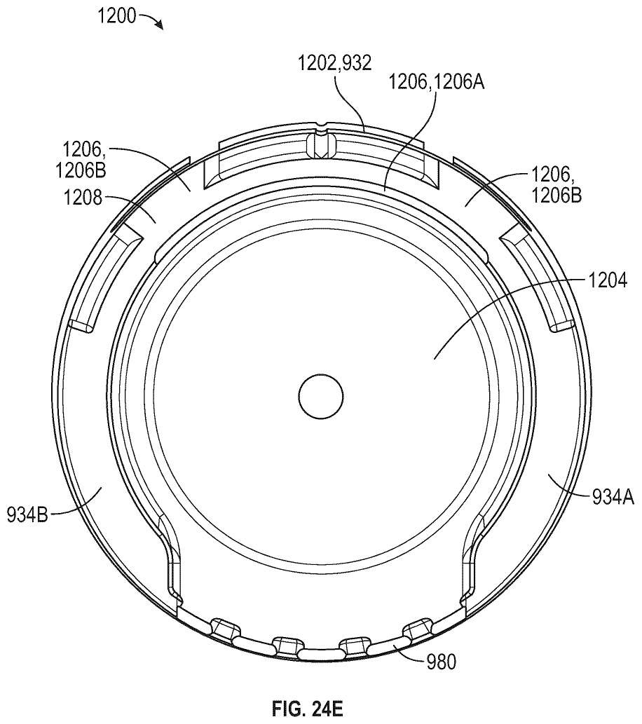

FIGS. 24A-24E show a trial component 1200. FIGS. 24A and 24B are perspective views of the trial component 1200. FIG. 24C is a plan view of a proximal side of the trial component 1200. FIG. 24D is a plan view of a medial side of the trial component 1200. FIG. 24E is a plan view of a distal side of the trial component 1200.

The trial component 1200 can be configured to simulate the configuration (shape, size, position, etc.) of the bearing 920 previously discussed in reference to FIGS. 19A-23. Thus, the trial component 1200 can be available in a plurality of standard sizes and shapes, for example. The trial component 1200 can be a temporary component that can be coupled to either the humeral tray 922 (see FIGS. 18A-18C and 20-23) previously discussed or a humeral tray trial. The trial component 1200 can be used for sizing the bearing 920 (previously shown) and/or to perform joint kinematics such as range of motion. The trial component 1200 differs from previous trials discussed herein in that it has one or more locking mechanisms 1202 rather than a plurality of such mechanisms. The one or more locking mechanisms 1202 can be separated from a main body 1204. The one or more locking mechanisms 1202 can be separated from the main body 1204 by a plurality of reliefs 1206, including interior reliefs 1206A and side reliefs 1206B. Bridges 1208 act as linkages to attach each of the one or more locking mechanisms 1202 to the main body 1204 and extend between the interior reliefs 1206A and the side reliefs 1206B. The one or more locking mechanisms 1202 can be flexed via the bridges 1208 outward to flex at least some of the plurality of tabs 932 outward. The one or more locking mechanisms 1202 can be located on a medial side of the trial component 1200, for example. The trial component 1200 can include other features shared with the bearing 920 (FIGS. 19A-23) including the plurality of fingers 980, the articular surface 928, the indicia 936, the relief(s) 934A and 934B and the plurality of tabs 932 as discussed previously.

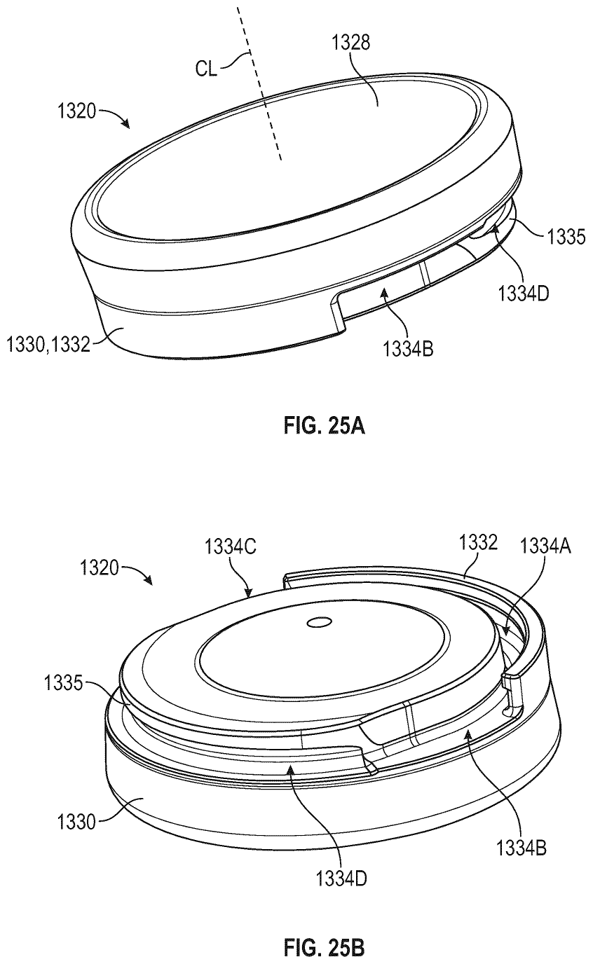

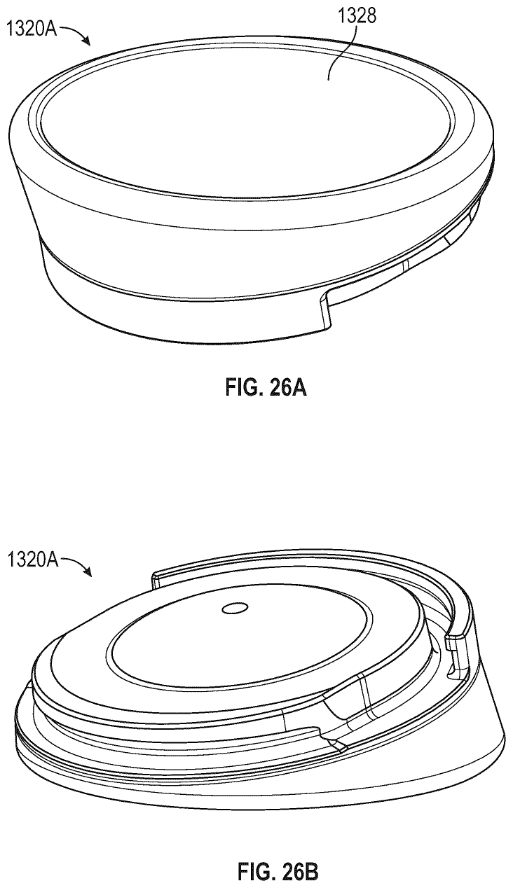

FIGS. 25A and 25B show an example of the bearing 1320. The bearing 1320 can include an articular surface 1328 (FIG. 25A), an outer edge 1330 or side, a single continuous tab 1332, a plurality of reliefs 1334A (FIGS. 25B), 1334B, 1334C (FIGS. 25B) and 1334D and a toe projection 1335.

The bearing 1320 can be formed of High Density Poly Ethylene (HDPE) or other suitable implantable material(s). The bearing 1320 can be available in a range of standard sizes including different thicknesses and diameters. The bearing 1320 can have a centerline axis CL (FIG. 25A). The articular surface 1328 of FIG. 25A can form a proximal most side of the bearing 1320 and can be cup or bowl shape to receive the ball component installed on the glenoid. The articular surface 1328 can be flat having edges with a relatively same height according to some examples. Thus, the bearing 1320 can have a generally same thickness from a medial side to a lateral side, for example. The articular surface 1328 can extend to adjacent the outer edge 1330. Outer edge 1330 can form the side of the bearing 1320. The outer edge 1330 can extend distally from at or adjacent the articular surface 1328 to the single continuous tab 1332 on a medial side and extending over 100 degrees of the medial side of the outer edge 1330. The single continuous tab 1330 can be formed by the relief 1334A along an interior side and can be shaped as a snap or lip with an overhanging feature such as a projection (e.g., a lip, barb or other feature) that extends into the relief 1334A. The single continuous tab 1332 can be configured to engage the humeral implant (not shown) as further described and shown herein. The single continuous tab 1332 can be circumferentially arranged along a distal part of the bearing 1320 forming a distal edge thereof.

The plurality of reliefs 1334B and 1334C (FIG. 25B) can communicate with the relief 1334A and can comprise gaps along the outer edge 1330. The plurality of reliefs 1334B and 1334C can form windows in the manner previously discussed. The plurality of reliefs 1334B and 1334C can extend along the circumference adjacent but extending inward of the outer edge 1330 to the relief 1334D. The relief 1334D can extend along the circumference along a lateral side of the outer edge 1330. The toe projection 1335 can be positioned distal of the relief 1334D and can form a distal portion of the relief 1334D. The toe projection 1335 can be an overhanging feature such as a projection (e.g., a lip, barb or other feature) that extends into the relief 1334D. The toe projection 1335 can be configured to engage the humeral implant (not shown) as further described and shown herein. The toe projection 1335 can be circumferentially arranged along a distal part of the bearing 1320 forming a distal edge thereof. The toe projection 1335 can be a single continuous locking feature on the lateral side and extending over 100 degrees of the lateral side of the outer edge 1330.

FIGS. 26A and 26B show another example of a bearing 1320A constructed in the manner of the bearing 1320 of FIGS. 25A and 25B but differs in having an articular surface 1328A (FIG. 26A) can be angled or tilted according to some examples. Thus, the bearing 1320A can have a varying thickness or generally sloped thickness from a medial side to a lateral side, for example.

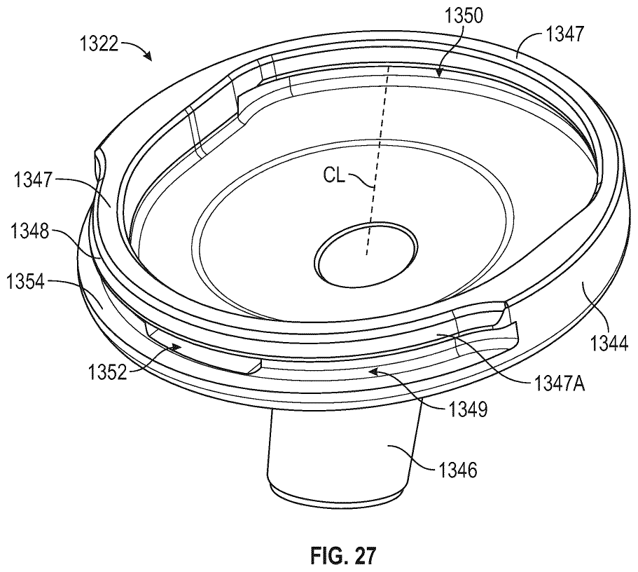

FIG. 27 shows a humeral tray 1322. The humeral tray 1322 can include a centerline axis CL, a main body 1344, a stem 1346, a rail 1347, a barb 1348, a relief 1350, a insertion slot 1352 and a skirt 1354.

The main body 1344 can be generally dish shaped or otherwise curved and configured to receive distal parts of the bearing (not shown). The stem 1346 can extend distal of the main body 1344 and can be configured with a taper or other mechanical feature to couple with the humeral stem (not shown). The rail 1347 or other feature can extend from the main body 1344 and can form an outer proximal edge of the humeral tray 1322. The rail 1347 can include a chamfered surface 1347A configured to be engaged by a chamfered surface of the bearing 1320 or 1320A.

The barb 1348 can project outward from the rail 1347. The barb 1348 can project generally outward from the centerline axis CL of the humeral tray 1322. The barb 1348 can extend around less than an entirety of a circumference of the humeral tray 1322. The barb 1348 can comprise an overhanging feature of the rail 1347, for example. The barb 1348 can form a portion of a groove 1349.

The groove 1349 can extend around less than a majority of the circumference of the humeral tray 1322 distal of the barb 1348 on a medial side of the humeral tray 1322. The insertion slot 1352 can extend through the rail 1347 on the medial side distal of the barb 1348. The slot 1352 can communicate with an interior of the humeral tray 1322 and can communicate with the groove 1349. The relief 1350 can be on an opposing lateral side of the humeral tray 1322 from the groove 1349. The relief 1350 can be an undercut or similar feature of the rail 1347 configured to receive the toe projection 1335 (FIGS. 25A and 25B) of the bearing. The skirt 1354 can be positioned distal of the groove 1349 and can form a portion thereof. The skirt 1354 can project radially outward of the groove 1349, and indeed, radially outward of the barb 1348 and rail 1347.

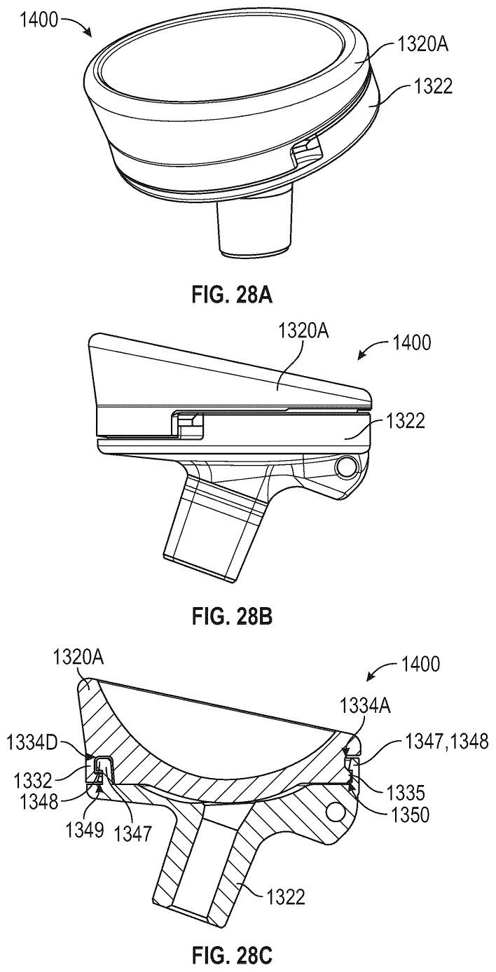

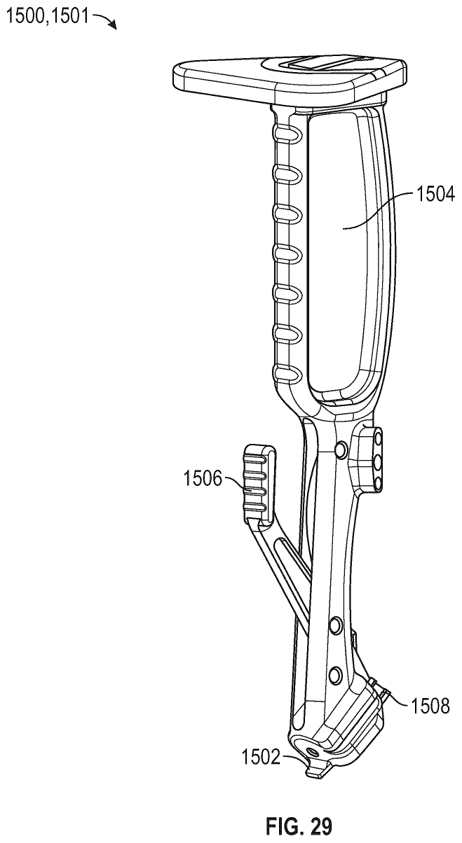

FIGS. 28A-28C show an orthopedic assembly 1400 of the humeral tray 1322 and the bearing 1320A. The bearing 1320A is coupled to the humeral tray 1322A. Coupling can be accomplished in a manner similar to the techniques described in FIGS. 9A-11A and 23, for example with the single continuous tab 1332 (FIG. 28C) flexing outward over the barb 1348 and the rail 1347. FIG. 28C shows the relief 1334D receiving parts of the rail 1347 and parts of the barb 1348 therein. The groove 1349 receives the single continuous tab 1332. A tool (illustrated in FIG. 29) can be inserted through the relief 1334D and the rail 1347 via the insertion slot 1352 (not shown in FIGS. 28A-28C). The insertion slot 1352 (not shown in FIGS. 28A-28C) is configured to provide access to the tool for insertion of the tool between the bearing 1320A and the humeral tray 1322 for assembly of the bearing 1320A with the humeral tray 1322. As shown in FIG. 28C, the toe projection 1335 and relief 1334A are configured to receive or be received by the rail 1347, the barb 1348 and the relief 1350 on the lateral side of the assembly 1400.

As shown in FIGS. 28A-28C the outer edge 1330 along the single continuous tab 1332 is positioned radially inward of or flush with an outer edge of the skirt 1354 when the bearing 1320A is coupled to the humeral tray 1322. The skirt 1354 can extend to protect an interior of the single continuous tab 1332, the barb 1348 and the groove 1349 (together comprising a locking mechanism as shown in FIG. 28C) from contact with tissue.

FIG. 29 shows a tool 1500, an inserter 1501, for coupling the humeral tray to the bearing to form an assembly such as the orthopedic assembly 1400 of FIGS. 27A-27C. The tool 1500 can include a projection tab 1502 configured for insertion into the insertion slot 1352 (FIG. 27). The tool 1500 can include a handle 1504, a lever 1506 and a slide 1508. The slide 1508 can be actuated by the lever 1506 and action of an internally loaded spring (not shown). The slide 1508 via the spring and lever 1506 can force down the bearing onto the humeral tray.

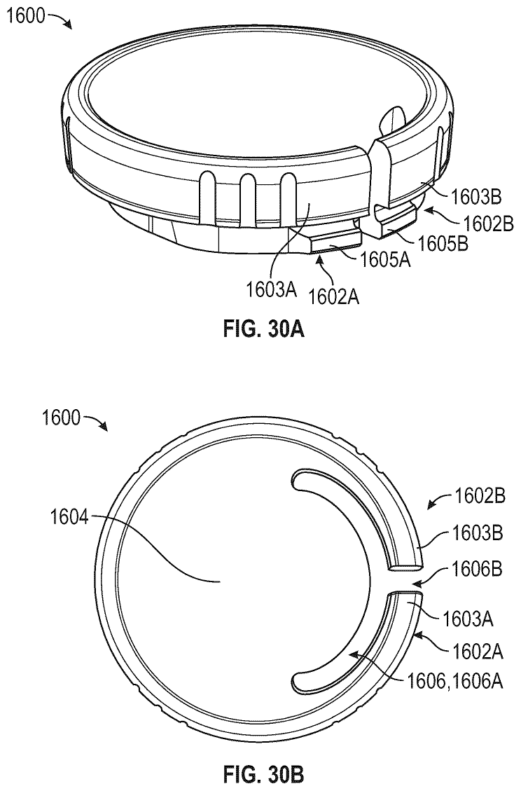

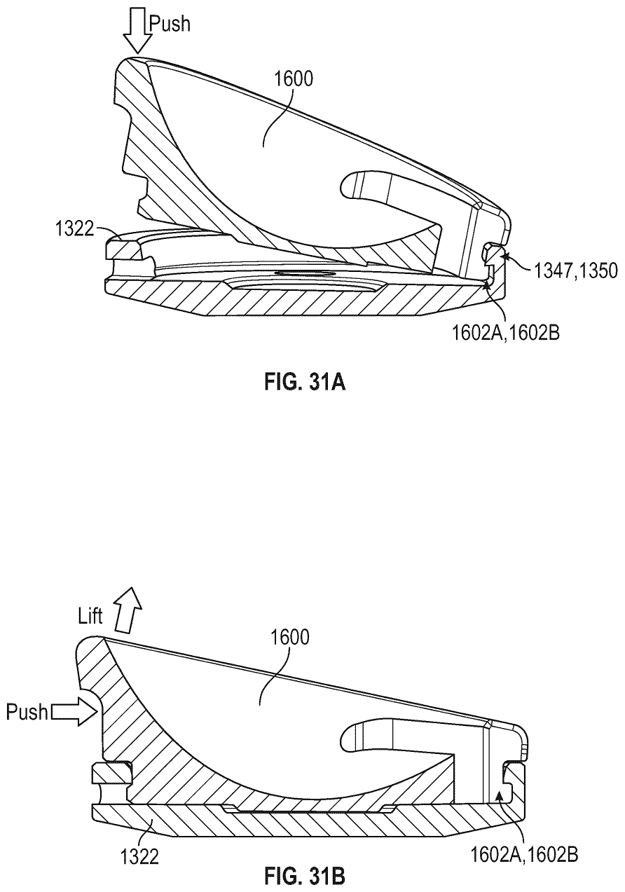

FIGS. 30A and 30B show an example of a trial component 1600 that can be configured to simulate the configuration (shape, size, position, etc.) of the bearing 1320 or 1320A previously discussed in reference to FIGS. 25A-27B. Thus, the trial component 1600 can be available in a plurality of standard sizes and shapes, for example. The trial component 1600 can be a temporary component that can be coupled to either the humeral tray 1322 (see FIGS. 31A and 31B) or a humeral tray trial. The trial component 1600 can be used for sizing the bearing 1320 or 1320A (previously shown) and/or to perform joint kinematics such as range of motion. The trial component 1600 differs from previous trials discussed herein in that it has two separated locking mechanisms 1602A and 1602B. The two locking mechanisms 1602A and 1602B comprise arms 1603A and 1603B having finger projections 1605A and 1605B (shown only in FIG. 30A), respectively.

As best shown in FIG. 30B, the arms 1603A and 1603B can be separated from a main body 1604. The locking mechanisms 1602A and 1602B can be separated from the main body 1604 by a plurality of reliefs 1606, including interior reliefs 1606A and side relief 1606B. The locking mechanisms 1602A and 1602B via arms 1603A and 1603B can be flexed inward or outward to flex at least some of the finger projections 1605A and 1604 (FIG. 30A) as desired including in the manner shown in FIGS. 31A and 31B. The locking mechanisms 1602A and 1602B can be located on a lateral side of the trial component 1600, for example.

FIGS. 31A and 31B show a process of coupling (FIG. 31A) the trial component 1600 to and decoupling (FIG. 31B) the trial component 1600 from the humeral tray 1322. FIG. 31A shows the locking mechanisms 1602A and 1602B inserted in and engaging with the relief 1350 and the rail 1347 on the lateral side. The trial component 1600 is then forced generally distally downward onto the humeral tray 1322 as indicated by arrow in FIG. 31A. Locking mechanisms 1602A and 1602B are biased into engagement with the humeral tray 1322 during this process.

FIG. 31B shows the process of removing the trial component 1600 from the humeral tray 1322. This includes pushing (as indicated by arrow) on a medial side of the trial component 1600 in order to flex the locking mechanisms 1602A and 1602B further inward, this allows the medial portion of the trial component 1600 to be lifted upward (as indicated by arrow) for disengaging the medial side of the trial component from the humeral tray 1322.

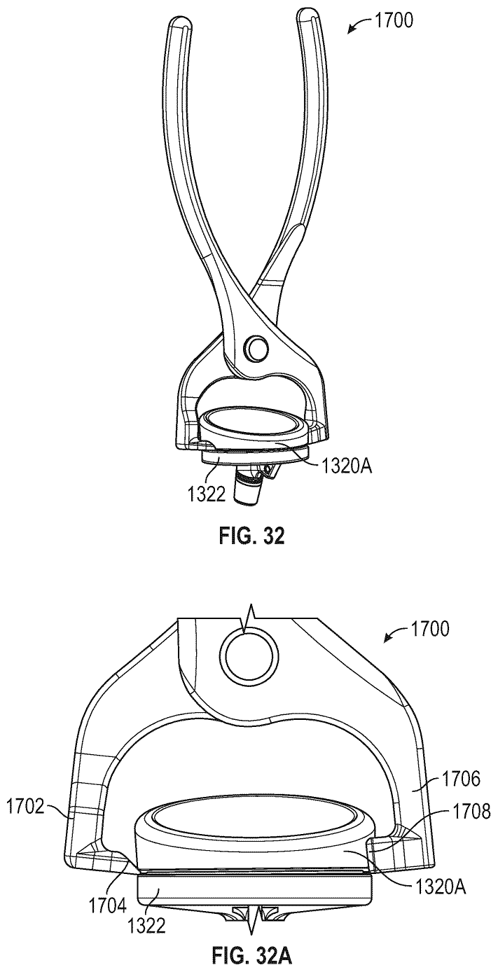

FIGS. 32 and 32A show a tool 1700 configured for facilitating removal of the bearing 1320A from the humeral tray 1322. As shown in FIG. 32A, the tool 1700 can comprise a pliers like component having a first jaw 1702 with a wedge tip 1704 component and a second jaw 1706 with a blunt tip 1708. The wedge tip 1704 can be forced between the bearing 1320A and the humeral tray 1322 by action of the tool 1700 including closing of the first jaw 1702 and the second jaw 1706.

The term “proximal” refers to the general orientation of the side and/or surface when the humeral implant is implanted in the bone. Thus, “proximal” refers to a direction or location generally in the direction of or toward the head of a patient, and “distal” refers to the opposite direction of proximal, i.e., away from the head of a patient. As used herein, the terms “anterior” and “posterior” should be given their generally understood anatomical interpretation. Thus, “posterior” refers to a location or direction generally toward a rear of the patient. Similarly, “anterior” refers to a location or direction generally toward a front of the patient. Thus, “posterior” refers to the opposite direction of “anterior.” Similarly, the terms “medial” and “lateral” should be given their generally understood anatomical interpretation. “Medial” refers to the more inward facing (inner part) of the prosthesis or guide (when in the implanted orientation) and “lateral” refers to the outer part or outward facing part. “Medial” refers to the opposite direction of “lateral.” The terms “substantially”, “generally” or “about” means within 10% of the value discussed.

It will be readily understood to those skilled in the art that various other changes in the details, material, and arrangements of the parts and method stages which have been described and illustrated in order to explain the nature of the inventive subject matter can be made without departing from the principles and scope of the inventive subject matter as expressed in the subjoined claims. For example, the order of method steps or stages can be altered from that described above, as would be appreciated by a person of skill in the art.

It will also be appreciated that the various dependent claims, examples, and the features set forth therein can be combined in different ways than presented above and/or in the initial claims. For instance, any feature(s) from the above examples can be shared with others of the described examples, and/or a feature(s) from a particular dependent claim may be shared with another dependent or independent claim, in combinations that would be understood by a person of skill in the art.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A prosthesis assembly for a reverse shoulder arthroplasty comprising:
a bearing having a tab arranged along at least a first part of an outer edge thereof and a toe projection arranged along a second part of an outer edge thereof; and
a humeral implant having a wall with a barb and an insertion slot passing through the wall at a location distal of the barb, wherein the insertion slot is configured to receive a portion of an insertion tool therein, and wherein a portion of the barb engages with the tab coupling the bearing with the humeral implant.

2. The prosthesis assembly of claim 1, wherein the bearing includes at least a first relief and a second relief, wherein the first relief is configured to receive the toe projection adjacent the tab, and wherein the second relief is positioned outward of and proximal of the toe projection and is configured to receive the portion of the barb therein.

3. The prosthesis assembly of claim 2, wherein the bearing that includes tab forms a medial side of the bearing, and wherein the toe projection is arranged along a lateral side of the bearing.

4. The prosthesis assembly of claim 3, wherein the humeral implant includes a groove along a medial side thereof, wherein the insertion slot communicates with the groove.

5. The prosthesis assembly of claim 4, wherein the humeral implant includes a skirt positioned distal of the groove, wherein the skirt projects outward of the groove away from a centerline axis of the humeral implant, wherein the outer edge of the bearing along at least the first part is substantially aligned with or positioned inward of the skirt.

6. The prosthesis assembly of claim 5, wherein an opposing wall of the humeral implant on the lateral side includes a relief forming an undercut configured to receive the toe projection therein.

7. A prosthesis assembly for a reverse shoulder arthroplasty comprising:
a bearing having a tab arranged along at least a first part of an outer edge thereof; and
a humeral implant having a wall with a barb and a skirt positioned distal of the barb, wherein the skirt projects outward of the tab away from a centerline axis of the humeral implant, wherein the tab is substantially aligned with or positioned inward of the skirt, and wherein a portion of the barb engages with the tab coupling the bearing with the humeral implant.

8. The prosthesis assembly of claim 7, further comprising an insertion slot passing through the wall at a location distal of the barb, wherein the insertion slot is configured to receive a portion of an insertion tool therein, and wherein a portion of the barb engages with the tab coupling the bearing with the humeral implant.

9. The prosthesis assembly of claim 8, further comprising a toe projection arranged along a second part of an outer edge thereof.

10. The prosthesis assembly of claim 9, wherein the bearing includes at least a first relief and a second relief, wherein the first relief is configured to receive the toe projection adjacent the tab, and wherein the second relief is positioned outward of and proximal of the toe projection and is configured to receive the portion of the barb therein.

11. The prosthesis assembly of claim 7, wherein the humeral implant includes a groove along a medial side thereof, wherein the skirt is positioned distal of the groove, wherein the skirt projects outward of the groove away from a centerline axis of the humeral implant.

12. A prosthesis assembly for a reverse shoulder arthroplasty comprising:
a trial component configured to simulate a bearing, wherein the trial component has two coupling mechanisms including two arms each including at least one finger along an outer edge thereof, each of two arms separated by a relief, and
a humeral implant configured to receive and couple with the trial component.

13. The prosthesis assembly of claim 12, wherein the humeral implant has a wall with a barb and an insertion slot passing through the wall at a location distal of the barb.

14. The prosthesis assembly of claim 13, wherein the humeral implant includes a groove along a medial side thereof adjacent the wall and barb, wherein the insertion slot communicates with the groove.

15. The prosthesis assembly of claim 14, wherein the humeral implant includes a skirt positioned distal of the groove, wherein the skirt projects outward of the groove away from a centerline axis of the humeral implant, wherein an outer edge of the trial component is substantially aligned with or positioned inward of the skirt.

16. The prosthesis assembly of claim 15, wherein an opposing wall of the humeral implant on a lateral side includes a relief forming an undercut configured to receive the two coupling mechanisms therein.

17-50. (canceled)
